(19) United States
(12) Patent Application Publication
(10) Pub. No.: US 2026/0200882 A1
(43) Pub. Date: Jul. 16, 2026

(54) NOVEL FIVE-MEMBERED HETEROCYCLE SUBSTITUTED STYRENE DERIVATIVE, AND PREPARATION METHOD THEREFOR AND USE THEREOF

(71) Applicant: Guangzhou Nanocrystal Biotechnology Co., Ltd., Guangzhou, Guangdong (CN)

(72) Inventors: Huixiong CHEN, Guangzhou, Guangdong (CN); Yongliang LI, Guangzhou, Guangdong (CN); Shanpeng CHU, Guangzhou, Guangdong (CN); Yehao LIANG, Guangzhou, Guangdong (CN); Ziheng YE, Guangzhou, Guangdong (CN); Bankang RUAN, Guangzhou, Guangdong (CN)

(73) Assignee: Guangzhou Nanocrystal Biotechnology Co., Ltd., Guangzhou, Guangdong (CN)

(21) Appl. No.: 19/134,736

(22) PCT Filed: Jun. 21, 2023
(86) PCT No.: PCT/CN2023/101587

§ 371 Date: Jun. 1, 2025

(30) Foreign Application Priority Data
Jun. 21, 2022 (CN) 202210705821.X

(51) Int. Cl. C07D 307/42; A61K 31/341; A61K 31/381; A61K 31/426; A61P 29/00; C07D 277/24; C07D 333/16

(52) U.S. Cl. CPC C07D 307/42; A61K 31/341; A61K 31/381; A61K 31/426; A61P 29/00; C07D 277/24; C07D 333/16

(57) ABSTRACT

The present application relates to the technical field of biological medicines, and in particular relates to a novel five-membered heterocycle substituted styrene derivative, and a preparation method therefor and the use thereof. The styrene derivative has a novel structure and a significant anti-inflammatory effect. A new treatment method and a new anti-inflammatory drug molecule is provided for solving the problems of a series of adverse reactions, tolerance, side effects, etc., being easily generated due to the long-term use of a large number of anti-inflammatory drugs.

This application claims the priority of the Chinese patent application with the application number of 202210705821.X and of the title of “Novel five-membered heterocycle substituted styrene derivative, and preparation method therefor and use thereof” before the CNIPA, China National Intellectual Property Administration filed on Jun. 21, 2022, which is incorporated herein in its entirety by reference.

### TECHNICAL FIELD

The present application relates to the technical field of biological medicines, and in particular relates to a novel five-membered heterocycle substituted styrene derivative, and a preparation method therefor and a use thereof.

### BACKGROUND ART

Inflammation is a defense response of a body to stimulation, manifested as redness, swelling, heat, pain and dysfunction. As an important pathological process, inflammation is very common in a human body. Most diseases are accompanied by the inflammatory mediators and the inflammatory responses, which attack its own tissues of the body and increase the damage of the diseases to the body, thereby leading to cancer, acute lung injury, diabetes complications, rheumatoid arthritis, psoriasis, atopic dermatitis, inflammatory bowel disease and other diseases.

Inflammatory lesions are mainly localized, but the local lesions can affect the entire body. In relatively serious inflammatory diseases, obvious systemic reactions often occur. Inflammatory reactions are a pathogenetic basis of some diseases. Severe hypersensitivity reactions can threaten the lives of patients when the inflammation becomes too intense.

The long-term and excessive use of anti-inflammatory drugs easily generates a series of adverse reactions, tolerance, side effects, etc. Therefore, the search for anti-inflammatory compounds with significant anti-inflammatory effect and low side effects is still a research hotspot in the field of medicinal chemistry.

### SUMMARY OF THE INVENTION

In view of this, the present application provides a novel five-membered heterocycle substituted styrene derivative, and a preparation method therefor and a use thereof. The five-membered heterocycle substituted styrene derivative with novel chemical structure has a significant anti-inflammatory effect, which provides a new treatment method and a new anti-inflammatory drug molecule for solving the problems of a series of adverse reactions, tolerance, side effects, etc., due to the long-term and excessive use of anti-inflammatory drugs.

A first aspect of the present application provides a novel five-membered heterocycle substituted styrene derivative having a structure shown in formula (I) or a pharmaceutically acceptable salt thereof, a solvent compound of the pharmaceutically acceptable salt, an enantiomer, a diastereoisomer, a tautomer, a despinner or a combination thereof;

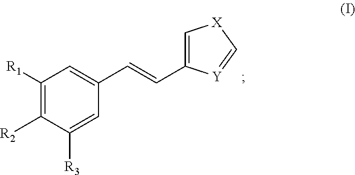

in formula (I), R₁ and R₃ are selected from H, OH, alkoxy or acyl; R₂ is selected from H, alkyl, cycloalkyl, alkenyl, cycloalkenyl, aralkyl or halogen; X is selected from O, S or NR₄, and R₄ is selected from H or alkyl; and Y is selected from C or N.

Preferably, the pharmaceutically acceptable salts include hydrochloride, sulfate, nitrate, phosphate, metaphosphate, mesylate, esilate, citrate, besylate, p-methyl benzenesulfonate, malate, tartrate, succinate, fumarate, acetate, hydroxyacetate, isethionate, maleate, lactate, lactobionate or trifluoroacetate.

Preferably, the R₁ is selected from OH; the R₃ is selected from OH; the R₂ is selected from propyl; X is selected from O; and Y is selected from C.

Preferably, the R₁ is selected from OH; the R₃ is selected from OH; the R₂ is selected from propyl; X is selected from S; and Y is selected from N.

Preferably, the R₁ is selected from OH; the R₃ is selected from OH; the R₂ is selected from cyclopropyl; X is selected from S; and Y is selected from C.

Preferably, the R₁ is selected from OH; the R₃ is selected from OH; the R₂ is selected from cyclopropyl; X is selected from O; and Y is selected from C.

Preferably, the R₁ is selected from OH; the R₃ is selected from OH; the R₂ is selected from cyclopent-1-ene-1-yl; X is selected from S; and Y is selected from C.

Preferably, the R₁ is selected from OH; the R₃ is selected from OH; the R₂ is selected from cyclopent-1-ene-1-yl; X is selected from S; and the Y is selected from N.

Preferably, the R₁ is selected from OH; the R₃ is selected from OH; the R₂ is selected from cyclopentyl; X is selected from S; and Y is selected from C.

Preferably, the R₁ is selected from OH; the R₃ is selected from OH; the R₂ is selected from bromo; X is selected from O; and Y is selected from C.

Preferably, the R₁ is selected from OH; the R₃ is selected from OH; the R₂ is selected from prop-1-ene-2-yl; X is selected from S; and Y is selected from C.

Preferably, the R₁ is selected from OH; the R₃ is selected from OH; the R₂ is selected from bromo; X is selected from S; and Y is selected from C.

Preferably, the R₁ is selected from OH; the R₃ is selected from OH; the R₂ is selected from prop-1-ene-2-yl; X is selected from O; and Y is selected from C.

Preferably, the R₁ is selected from OH; the R₃ is selected from OH; the R₂ is selected from prop-1-ene-2-yl; X is selected from S; and Y is selected from N.

Preferably, the R₁ is selected from OH; the R₃ is selected from OH; the R₂ is selected from cyclopropyl; X is selected from S; and Y is selected from N.

Preferably, the R₁ is selected from OH; the R₃ is selected from methoxy; the R₂ is selected from bromo; X is selected from S; and Y is selected from C.

A second aspect of the present application provides a preparation method for the five-membered heterocycle substituted styrene derivative, including steps:
step 1: under the nitrogen protection and the action of catalyst tetrabutylammonium bromide, a styrene derivative intermediate is substituted with triethyl phosphite to obtain the styrene derivative with an ethyl phosphite group;
step 2: under the nitrogen protection and the action of alkali reagent sodium hydride, making the styrene derivative with the ethyl phosphite group and a five-membered heterocycle conduct a second substitution reaction to obtain a five-membered heterocycle substituted styrene derivative intermediate;
step 3: under the action of aluminum trichloride and N,N-dimethylaniline, removing a benzyl functional group on the five-membered heterocycle substituted styrene derivative intermediate to obtain the five-membered heterocycle substituted styrene derivative;
the styrene derivative intermediate is 3,5-dibenzoxy-4-isopropylbenzyl bromide, 3,5-dimethoxy-4-isopropylbenzyl bromide, 5-(bromomethyl)-2-cyclopropyl-1,3-dimethoxybenzene, 5-(bromomethyl)-2-(cyclopent-1-ene-1-yl)-1,3-dimethoxybenzene, 5-(bromomethyl)-2-cyclopentyl-1,3-dimethoxybenzene, 2-bromo-5-(bromomethyl)-1,3-dimethoxybenzene or 5-(bromomethyl)-1,3-dimethoxy-2-(prop-1-ene-2-yl)benzene;
the five-membered heterocycle is 4-furfural, thiazole-5-formaldehyde, 3-thenaldehyde, 3-furfural, thiazole-4-formaldehyde, 3,5-dimethoxy-4-(1-methylvinyl)benzaldehyde or 3-methylfuran;
the temperature of the first substitution reaction is 100° C.-150° C., and the time is 4 h-8 h;
the temperature of the second substitution reaction is 20° C.-30° C., and the time is 8 h-16 h.

A third aspect of the present application provides a use of the novel five-membered heterocycle substituted styrene derivatives.

It should be noted that the styrene derivatives derived from natural plant ingredients are widely distributed in nature, have many biological activities, and show unique pharmacological effects, including anti-cancer, anti-inflammatory, antibacterial, lipid-lowering, anti-Alzheimer's, antioxidant, and treatment of immune diseases and other important biological functions. They have good water solubility and few side effects, and can also be used as anti-inflammatory drugs. To sum up, the present application provides a novel five-membered heterocycle substituted styrene derivative, and a preparation method therefor and a use thereof, wherein the five-membered heterocycle substituted styrene derivative is tested for toxicity to mouse macrophages and the inhibition effect of lipopolysaccharide-induced nitric oxide production in macrophages. The results show that the five-membered heterocycle substituted styrene derivatives with novel chemical structures have low side effects, and good anti-inflammatory activities, and are new anti-inflammatory compounds, which can be used as new anti-inflammatory drugs.

### DETAILED DESCRIPTION OF THE INVENTION

To make the invention purpose, features and advantages of the present application more clear and understandable, the technical solutions in embodiments of the present invention will be clearly and fully described below. Apparently, the embodiments described below are merely part of the embodiments of the present application, not all of the embodiments. Based on the embodiments in the present application, all other embodiments obtained by the person having ordinary skill in the art without contributing creative labor shall belong to the protection scope of the present application.

### Embodiment 1

The present embodiment 1 provides preparation methods for styrene derivative intermediates (6a, 6b, 6c, 6d, 6e and 6f).

A synthetic route of 3,5-dibenzoxy-4-isopropylbenzyl bromide (6a) is as follows:

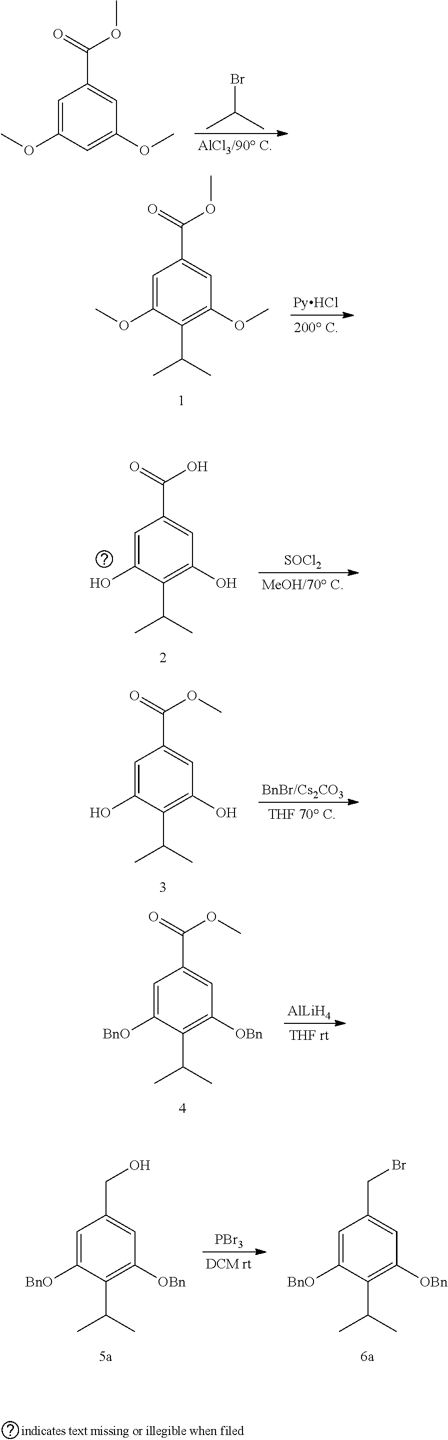

The preparation method for 3,5-dibenzoxy-4-isopropylbenzyl bromide includes steps:

### Step 1: 3,5-dimethoxy-4-isopropyl Methyl Benzoate (1) was Prepared

3,5-dimethoxy-methyl benzoate (4 g, 20.4 mmol, 1 eq) and 2-bromopropane (2.76 g, 22.4 mmol, 1.1 eq) were weighed and dissolved in 1,2-dichloroethane (20 mL). Anhydrous aluminum trichloride (2.98 g, 22.4 mmol, 1.1 eq) was added while stirring, and the mixture was heated to 90° C. and stirred for 6 h. After the completion of the reaction under TLC monitoring, the mixture was poured into a 500 mL beaker; 200 mL of saturated sodium bicarbonate solution was added, and filtered. The filtrate was extracted with ethyl acetate. The organic solution was dried with magnesium sulfate, and then concentrated under reduced pressure. A silica gel column was used for the separation of product 1 (2.91 g), which was a light yellow solid with a melting point of 106° C. and a yield of 60%. ¹H NMR (400 MHz, CDCl₃) δ 7.22 (s, 2H), 3.90 (s, 3H), 3.85 (s, 6H), 3.63 (hep, 1H), 1.27 (d, J=7.2 Hz, 6H); GC-MS=238.

### Step 2: 3,5-dihydroxy-4-isopropyl Benzoic Acid (2) was Prepared

The compound 1 (2 g, 8.4 mmol, 1 eq) and pyridine hydrochloride (3.88 g, 33.6 mmol, 4 eq) were weighed and placed in a 50 mL round-bottomed flask to react at 200° C. for 6 h. After the completion of the reaction under TLC monitoring, 10 mL of ice water was slowly added and the reaction solution was extracted with ethyl acetate after cooling. An organic phase was dried with magnesium sulfate, and then concentrated under reduced pressure. A silica gel column was used for the separation of product 2 (1.28 g), which was a light yellow solid with a melting point of 183° C. and a yield of 78%, ¹H NMR (400 MHz, DMSO) δ 12.41 (s, 1H), 9.31 (s, 2H), 6.89 (s, 2H), 3.47 (hep, 1H), 1.24 (d, J=7.2 Hz, 6H); GC-MS=196.

### Step 3: 3,5-dihydroxy-4-isopropyl Methyl Benzoate (3) was Prepared

The compound 2 (1.96 g, 10 mmol, 1 eq) was weighed and dissolved in a methanol (10 mL) solution. Thionyl chloride (1.78 g, 15 mmol, 1.5 eq) was added dropwise and heated to 70° C. to react for 4 h. After the completion of the reaction under TLC monitoring, the product was concentrated under reduced pressure. A silica gel column was used for the separation of product 3 (1.78 g), which was a light yellow solid with a melting point of 151° C. and a yield of 85%, ¹H NMR (400 MHz, CDCl₃) δ 7.08 (s, 2H), 5.29 (s, 2H), 3.89 (s, 3H), 3.51 (hep, 1H), 1.28 (d, J=7.9 Hz, 6H); GC-MS=210.

### Step 4: 3,5-dibenzoxy-4-isopropyl Methyl Benzoate (4) was Prepared

The compound 3 (2.1 g, 10 mmol, 1 eq) and benzyl bromide (3.4 g, 20 mmol, 2 eq) were weighed and dissolved in 15 mL of tetrahydrofuran solution. Cesium carbonate (8 g, 25 mmol, 2.5 eq) was added to react at 70° C. for 12 h. After the completion of the reaction under TLC monitoring, the solvent was evaporated out under reduced pressure. The residue was dissolved in the ethyl acetate solution and washed with water. The organic phase was dried with magnesium sulfate, and concentrated under reduced pressure. A silica gel column was used for the separation of product 4 (3.47 g), which was a light yellow solid with a melting point of 127° C. and a yield of 89%. ¹H NMR (400 MHz, DMSO) δ 7.47 (d, J=7.2 Hz, 4H), 7.43 (t, J=7.2 Hz, 4H), 7.34 (t, J=7.2 Hz, 2H), 7.30 (s, 2H), 5.17 (s, 4H), 3.84 (s, 3H), 3.66 (hep, 1H), 1.24 (d, J=7.2 Hz, 6H); GC-MS=390.

### Step 5: 3,5-dibenzoxy-4-isopropyl Benzyl Alcohol (5a) was Prepared

The compound 4 (3.71 g, 10.2 mmol, 1 eq) was weighed and dissolved in an anhydrous tetrahydrofuran (10 mL) solution. Lithium aluminum hydride (0.49 g, 12.24 mmol, 1.2 eq) was added at 0° C. while stirring to react at room temperature for 6 h. After the completion of the reaction under TLC monitoring, the solvent was evaporated out under reduced pressure. The residue was dissolved in a dichloromethane solution and washed with water. The organic phase was dried on MgSO₄ and concentrated under reduced pressure. A silica gel column was used for the separation of product 5a (2.96 g), which was a light yellow solid with a melting point of 87° C. and a yield of 80.4%, ¹H NMR (400 MHz, DMSO) δ 7.48 (d, J=7.8 Hz, 4H), 7.41 (t, J=7.8 Hz, 4H), 7.33 (t, J=7.8 Hz, 2H), 6.70 (s, 2H), 5.07 (s, 4H), 4.42 (d, J=3.6 Hz, 2H), 3.61 (hep, 1H), 1.22 (d, J=7.2 Hz, 6H); GC-MS=362.

### Step 6: 3,5-dibenzoxy-4-isopropylbenzyl Bromide (6a) was Prepared

The compound 5a (3.62 g, 10 mmol, 1 eq) was weighed and dissolved in 10 mL of dichloromethane solution. Phosphorus tribromide (2 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for the separation of product 6a (3.32 g), which was a light yellow solid with a melting point of 74° C. in a yield of 78.4%, ¹H NMR (400 MHz, CDCl₃) δ 7.44 (d, J=7.1 Hz, 4H), 7.39 (t, J=7.4 Hz, 4H), 7.34 (t, J=7.2 Hz, 2H), 6.66 (s, 2H), 5.07 (s, 4H), 4.44 (s, 2H), 3.70 (hep, 1H), 1.29 (d, J=7.1 Hz, 6H); GC-MS=424.

A synthetic route of 3,5-dimethoxy-4-isopropylbenzyl bromide (6b) is as follows:

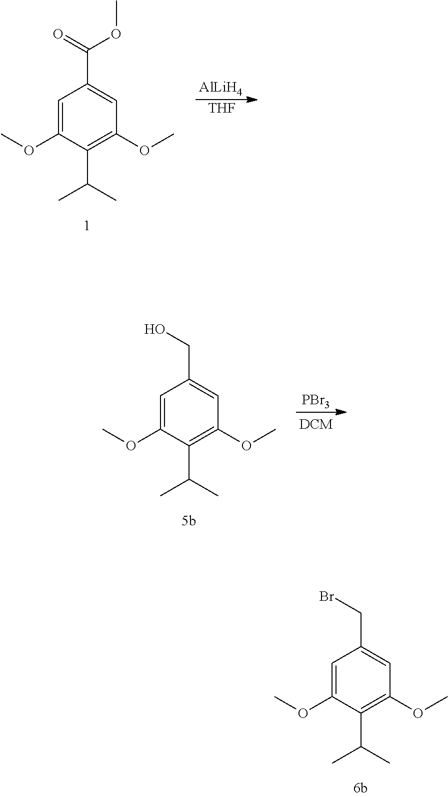

The preparation method for 3,5-dibenzoxy-4-isopropylbenzyl bromide (6b) includes steps:

### Step 1: 3,5-dimethoxy-4-isopropyl Benzyl Alcohol (5b) was Prepared

The compound 1 (1 g, 4.2 mmol, 1 eq) was weighed and dissolved in an anhydrous tetrahydrofuran (10 mL) solution. Lithium aluminum hydride (0.19 g, 5 mmol, 1.2 eq) was added while stirring at 0° C. and heated to room temperature to react for 6 h. After the completion of the reaction under TLC monitoring, the solvent was evaporated out under reduced pressure. The residue was dissolved in a dichloromethane solution and washed with water. The organic phase was dried with magnesium sulfate, and concentrated under pressure. A silica gel column was used for the separation of product 5b (0.74 g), which was a light yellow solid with a melting point of 95° C. and a yield of 84%, ¹H NMR (400 MHz, CDCl₃) δ 6.56 (s, 2H), 4.64 (d, J=3.6 Hz, 2H), 3.81 (s, 6H), 3.58 (hep, 1H), 1.27 (d, J=7.2 Hz, 6H); GC-MS=210.

### Step 2: 3,5-dibenzoxy-4-isopropylbenzyl Bromide (6b) was Prepared

The compound 5b (2.1 g, 10 mmol, 1 eq) was weighed and dissolved in 10 mL of dichloromethane. Phosphorus tribromide (2 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for the separation of product 6b (2.23 g), which was a light yellow solid with a melting point of 56° C. and a yield of 82%. ¹H NMR (400 MHz, CDCl₃) δ 6.56 (s, 2H), 4.46 (s, 2H), 3.80 (s, 6H), 3.56 (hep, 1H), 1.25 (d, J=7.2 Hz, 6H); GC-MS=272.

A synthetic route of 5-(bromomethyl)-2-cyclopropyl-1,3-dimethoxybenzene (6c) is as follows:

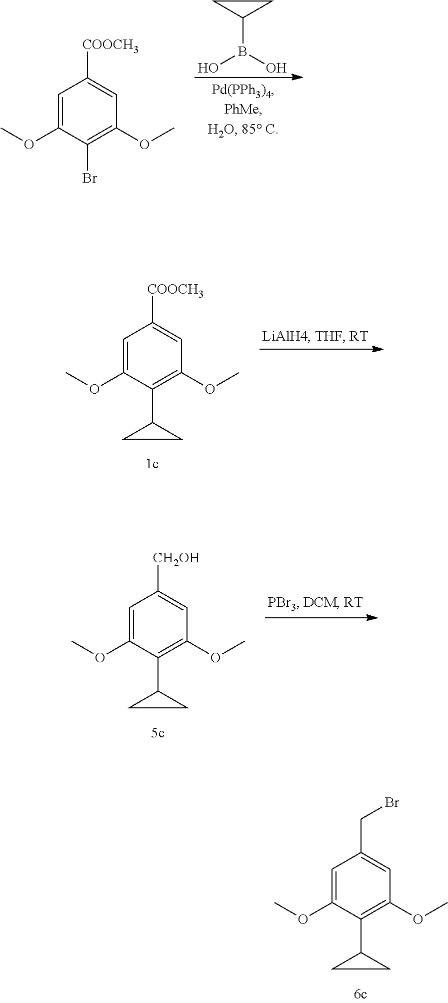

The preparation method for 5-(bromomethyl)-2-cyclopropyl-1,3-dimethoxybenzene (6c) includes steps:

### Step 1: 4-cyclopropyl-3,5-dimethoxy Methyl Benzoate (1c) was Prepared

4-bromo-3,5-dimethoxy methyl benzoate (2 g, 7.3 mmol) was weighed and dissolved in 25 ml of toluene, Cyclopropyl boric acid (1.2 g, 10.9 mmol, 1.5 eq), cesium carbonate (3.81 g, 11.5 mmol, 1.5 eq), tetrakis(triphenylphosphine) palladium (84 mg, 0.075 mmol, 0.01 eq) and water (176 mg) were added. The reaction was carried out at 100° C. for 12 h under nitrogen protection. After the completion of the reaction under TLC monitoring, the mixture was dissolved in ethyl acetate, and washed with water. The organic phase was dried on MgSO₄, and concentrated under reduced pressure. A silica gel column was used for the separation of product 6h (1.77 g), which was a white solid with a melting point and a yield of 85%.

### Step 2: 2-(4-cyclopropyl-3,5-dimethoxyphenyl) ethane-1-ol (5c) was Prepared

The compound 1c (1 g, 4.2 mmol, 1 eq) was weighed and dissolved in anhydrous tetrahydrofuran (10 mL). Lithium aluminum hydride (0.19 g, 5 mmol, 1.2 eq) was added while stirring at 0° C., and heated to room temperature to react for 6 h. After the completion of the reaction under TLC monitoring, the solvent was evaporated out under reduced pressure. The residue was dissolved in the dichloromethane solution and washed with water. The organic phase was dried with magnesium sulfate and concentrated under reduced pressure. A silica gel column was used for the separation of product 5c (0.74 g), which was a light yellow solid with a melting point of 95° C. and a yield of 84%.

Step 3: the compound 5c (2.1 g, 10 mmol, 1 eq) was weighed and dissolved in 10 mL of dichloromethane solution. Phosphorus tribromide (2 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for the separation of product 6c (2.23 g), which was a light yellow solid with a melting point of 56° C. in a yield of 82%.

A synthetic route of 5-(bromomethyl)-2-(cyclopent-1-ene-1-yl)-1,3-dimethoxybenzene (6d) is as follows:

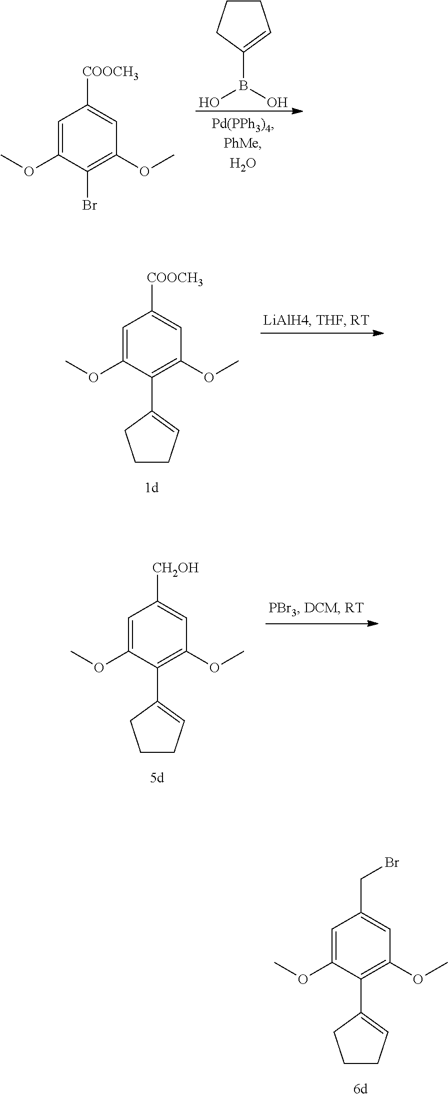

The preparation method for 5-(bromomethyl)-2-(cyclopent-1-ene-1-yl)-1,3-dimethoxybenzene includes steps:

### Step 1: 4-(cyclopent-1-ene-1-yl)-3,5-dimethoxy Methyl Benzoate (1d) was Prepared

4-bromo-3,5-dimethoxy methyl benzoate (2 g, 7.3 mmol) was weighed an dissolved in 25 ml of toluene; 1-cyclopentenylboronic acid (1.2 g, 10.9 mmol 1.5 eq). Potassium hydroxide (0.81 g, 14.5 mmol, 2 eq), tetrakis(triphenylphosphine) palladium (84 mg, 0.075 mmol, 0.01 eq) and water (176 mg) were added. The reaction was carried out at 100° C. for 12 h under nitrogen protection. After the completion of the reaction under TLC monitoring, the mixture was dissolved in ethyl acetate, and washed with water. The organic phase was dried on MgSO₄, and concentrated under pressure. A silica gel column was used for the separation of product 1d (1.6 g), which was a white solid with a melting point and a yield of 85%. ¹H NMR (400 MHz, CDCl₃) δ 7.24 (d, J=1.1 Hz, 2H), 5.85 (p, J=2.4 Hz, 1H), 3.92 (d, J=1.1 Hz, 3H), 3.83 (d, J=1.1 Hz, 6H), 2.65 (tq, J=7.4, 2.3 Hz, 2H), 2.60-2.48 (m, 2H), 1.99 (p, J=7.5 Hz, 2H); GC-MS=262.

### Step 2: (4-(cyclopent-1-ene-1-yl)-3,5-dimethoxyphenyl) methanol (5d) was Prepared

The compound 1d (1 g, 3.6 mmol, 1 eq) was weighed and dissolved in an anhydrous tetrahydrofuran (10 mL) solution. Lithium aluminum hydride (0.41 g, 10 mmol, 3 eq) was added while stirring at 0° C., and heated to room temperature to react for 6 h. After the completion of the reaction under TLC monitoring, the solvent was evaporated out under reduced pressure. The residue was dissolved in the dichloromethane solution and washed with water. The organic phase was dried with magnesium sulfate, and concentrated under reduced pressure. A silica gel column was used for the separation of product 5d (0.75 g), which was a light yellow solid with a melting point of 95° C. and a yield of 84%, ¹H NMR (400 MHz, MeOD) δ 6.63 (s, 2H), 5.66-5.60 (m, 1H), 4.59 (s, 2H), 3.77 (s, 6H), 2.61-2.51 (m, 2H), 2.47 (ddt, J=10.0, 4.8, 2.4 Hz, 2H), 1.95 (p, J=7.5 Hz, 2H); GC-MS=234.

### Step 3: 5-(bromomethyl)-2-(cyclopent-1-ene-1-yl)-1,3-dimethoxybenzene (6d) was Prepared

The compound 5d (0.75 g, 3 mmol, 1 eq) was weighed and dissolved in 10 mL of dichloromethane solution; phosphorus tribromide (2 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for separation to obtain a product 6d (817 mg), which was a light yellow solid with a melting point of 56° C. and a yield of 91%, ¹H NMR (400 MHz, CDCl₃) δ 6.59 (s, 2H), 5.77 (m, 1H), 4.48 (s, 2H), 3.79 (s, 6H), 2.61-2.51 (m, 2H), 2.52 (m, 2H), 1.98 (m, 2H); GC-MS=297.

A synthetic route of 5-(bromomethyl)-2-cyclopentyl-1,3-dimethoxybenzene (6e) is as follows:

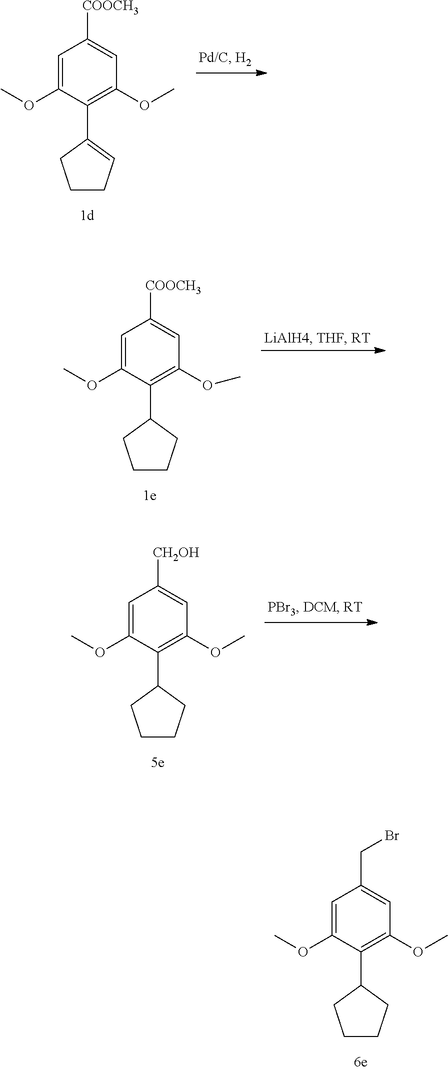

The preparation of 5-(bromomethyl)-2-cyclopentyl-1,3-dimethoxybenzene (6e) includes steps:

### Step 1: 4-cyclopentyl-3,5-dimethoxy Methyl Benzoate (1e) was Prepared

500 mg of compound 1d was added into 20 ml of methanol, and a palladium carbon catalyst was added. The reaction was carried out at room temperature for 24 h in a hydrogen atmosphere and palladium carbon was filtered out to obtain a crude product of white compound 1e. ¹H NMR (400 MHz, CDCl₃) δ 7.26 (s, 2H), 3.91 (s, 3H), 3.84 (s, 6H), 3.67 (p, J=8.9 Hz, 1H), 1.94-1.70 (m, 6H), 1.67-1.59 (m, 2H); GC-MS=264.

### Step 2: (4-cyclopentyl-3,5-dimethoxyphenyl) methanol (5e) was Prepared

The compound 1d (1 g, 3.6 mmol, 1 eq) was weighed and dissolved in anhydrous tetrahydrofuran (10 mL). Lithium aluminum hydride (0.41 g, 10 mmol, 3 eq) was added while stirring at 0° C., and heated to room temperature to react for 6 h. After the completion of the reaction under TLC monitoring, the solvent was evaporated out under reduced pressure. The residue was dissolved in the dichloromethane solution, and washed with water. The organic phase was dried with magnesium sulfate, and concentrated under reduced pressure. A silica gel column was used for the separation of product 5d (0.75 g), which was a light yellow solid with a melting point of 95° C. and a yield of 84%, ¹H NMR (400 MHz, CDCl₃) δ 6.59-6.53 (m, 2H), 4.63 (d, J=5.1 Hz, 2H), 3.83-3.77 (m, 6H), 3.61 (ddt, J=9.1, 6.3, 3.2 Hz, 1H), 1.92-1.83 (m, 2H), 1.81 (d, J=6.2 Hz, 2H), 1.77-1.69 (m, 2H), 1.62-1.58 (m, 2H); GC-MS=236.

### Step 3: 5-(bromomethyl)-2-cyclopentyl-1,3-dimethoxybenzene (6e) was Prepared

The compound 5d (0.75 g, 2.7 mmol, 1 eq) was weighed and dissolved in 10 mL of dichloromethane solution. Phosphorus tribromide (2 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for the separation of product 6e (817 mg), which was a light yellow solid with a melting point of 56° C. and a yield of 91%.

A synthetic route of 2-bromo-5-(bromomethyl)-1,3-dimethoxybenzene (6f) is as follows:

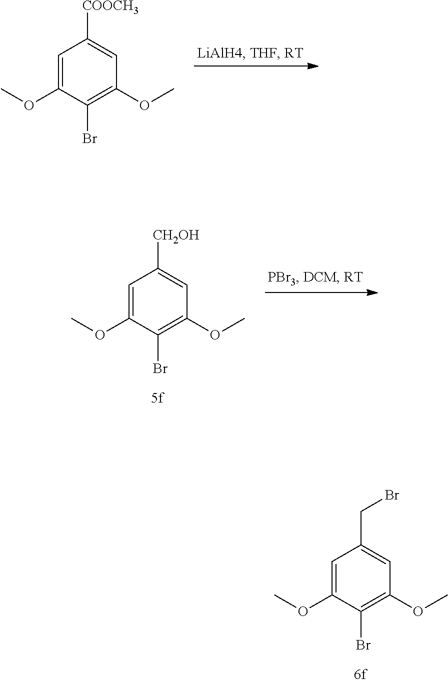

The preparation method for 2-bromo-5-(bromomethyl)-1,3-dimethoxybenzene (6f) includes steps:

### Step 1: 4-bromo-3,5-dimethoxybenzyl Alcohol (5f) was Prepared

The compound 4-bromo-3,5-dimethoxy methyl benzoate (1 g, 3.6 mmol, 1 eq) was weighed and dissolved in an anhydrous tetrahydrofuran (10 mL) solution. Lithium aluminum hydride (0.41 g, 10 mmol, 3 eq) was added while stirring at 0° C., and heated to room temperature to react for 6 h. After the completion of the reaction under TLC monitoring, the solvent was evaporated out under reduced pressure. The residue was dissolved in the dichloromethane, and washed with water. The organic phase was dried with magnesium sulfate and concentrated under reduced pressure. A silica gel column was used for the separation of product 5f (0.83 g), which was a light yellow solid with a melting point of 95° C. and a yield of 92%.

### Step 2: 2-bromo-5-(bromomethyl)-1,3-dimethoxybenzene (6f) was Prepared

The compound 5f (0.83 g, 3.1 mmol, 1 eq) was weighed and dissolved in 10 mL of dichloromethane. Phosphorus tribromide (1.6 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel columns was used for the separation of product of (897 mg), which was a light yellow solid with a melting point of 56° C. and a yield of 91%.

A synthetic route of 5-(bromomethyl)-1,3-dimethoxy-2-(prop-1-ene-2-yl)benzene (6g) is as follows:

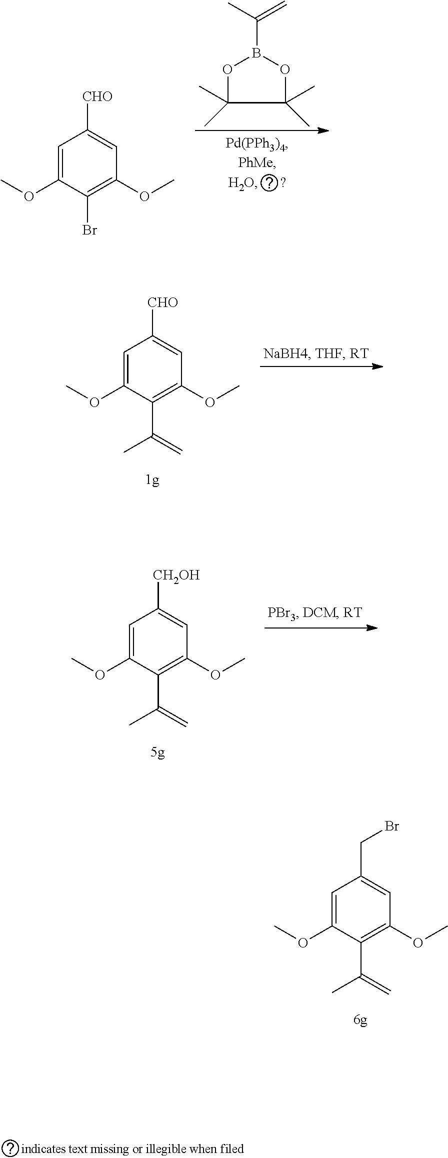

The preparation method for 5-(bromomethyl)-1,3-dimethoxy-2-(prop-1-ene-2-yl)benzene (6g) includes steps:

### Step 1: 3,5-dimethoxy-4-(1-methylvinyl)benzaldehyde (1g) was Prepared

4-bromo-3,5-dimethoxybenzaldehyde (2 g, 8.1 mmol) was weighed and dissolved in 25 mL of toluene. Isopropenyl pinacol ester (2.1 g, 12 mmol, 1.5 eq), sodium tert-butoxide (1.56 g, 16.3 mmol, 2 eq) and tetrakis(triphenylphosphine) palladium (94 mg, 0.08 mmol, 0.01 eq) were added. The reaction was carried out at 100° C. for 12 h under nitrogen protection. After the completion of the reaction under TLC monitoring, the mixture was dissolved in ethyl acetate and washed with water. The organic phase was dried on MgSO₄, and concentrated under reduced pressure. A silica gel column was used for the separation of product 1g (1.2 g), which was a white solid with a melting point of 79° C. and a yield of 75%. ¹H NMR (400 MHz, CDCl₃) δ 9.93 (s, 1H), 7.10 (s, 2H), 5.36 (t, J=1.7 Hz, 1H), 4.89 (dd, J=2.1, 1.0 Hz, 1H), 3.88 (s, 6H), 2.02 (t, J=1.3 Hz, 3H); GC-MS=261.

### Step 2: (3,5-dimethoxy-4-(prop-1-ene-2-yl)phenyl) methanol (5g) was Prepared

The compound 1g (1 g, 4.0 mmol, 1 eq) was weighed and dissolved in an anhydrous tetrahydrofuran (10 mL). Sodium borohydride (0.23 g, 6.0 mmol, 1.5 eq) was added while stirring at 0° C. and heated to room temperature to react for 12 h. After the completion of the reaction under TLC monitoring, ammonium chloride was added to stop the reaction. The product was extracted with ethyl ether. The organic phase was dried with magnesium sulfate, and concentrated under reduced pressure. A silica gel column was used for the separation of product 5g (0.83 g), which was a white solid with a melting point of 95° C. and a yield of 92%, ¹H NMR (400 MHz, CDCl₃) δ 6.62 (s, 2H), 5.38-5.32 (m, 1H), 4.88 (dd, J=2.3, 1.1 Hz, 1H), 4.70 (s, 2H), 3.84 (s, 6H), 2.03 (d, J=1.3 Hz, 3H); GC-MS=208.

### Step 3: 5-(bromomethyl)-1,3-dimethoxy-2-(prop-1-ene-2-yl)benzene (6g) was Prepared

The compound 5h (0.83 g, 3.9 mmol, 1 eq) was weighed and dissolved in 10 mL of dichloromethane. Phosphorus tribromide (1.6 mL) was added at 0° C. and heated to room temperature to react for 6 h. After the completion of the reaction under TLC monitoring, the reaction solution was washed with NaHCO₃ saturated aqueous solution. The organic phase was dried with magnesium sulfate, and concentrated under reduced pressure. A silica gel column was used for the separation of product 6g (897 mg), which was a light yellow solid with a melting point of 56° C. and a yield of 91%, ¹H NMR (400 MHz, CDCl₃) δ 6.63 (s, 2H), 5.35 (p, J=1.6 Hz, 1H), 4.88 (dd, J=2.3, 1.1 Hz, 1H), 4.51 (d, J=2.4 Hz, 2H), 3.84 (s, 3H); GC-MS=271.

### Embodiment 2

The present embodiment 2 provides a preparation method for the five-membered heterocycle substituted styrene derivative, with a synthetic route as follows:

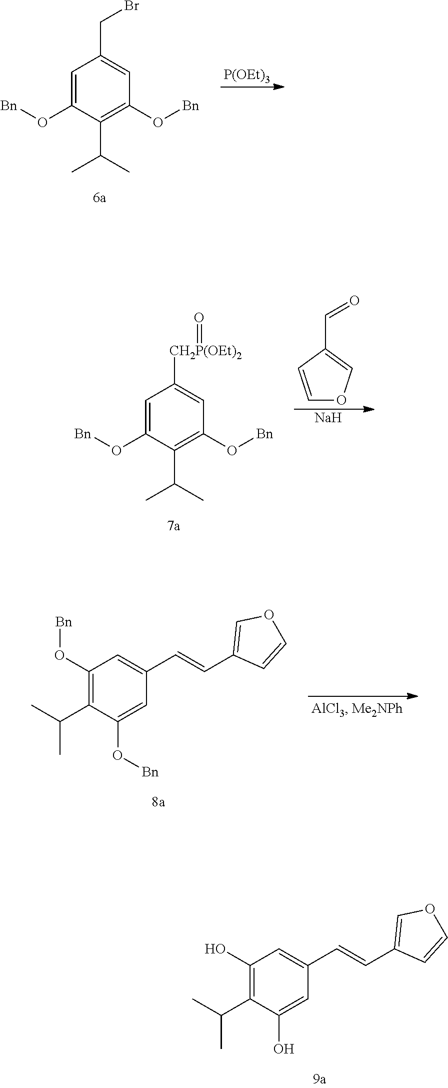

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6a (200 mg, 0.47 mmol, 1 eq) was weighed and added into a 25 mL round-bottomed flask. Triethyl phosphite (5 mL) was added. Tetrabutylammonium bromide (15 mg, 0.047 mmol, 0.1 eq) was added while stirring, and heated to 130° C. to react for 6 h under nitrogen protection and under heating at 80° C. The excess triethyl phosphite was removed under reduced pressure to obtain a crude product 7a.

### Step 2: A Styrene Derivative with an Ethyl Phosphite Group Undergoes a Second Substitution Reaction with a Five-Membered Heterocycle to Prepare a Five-Membered Heterocycle-Substituted Styrene Derivative Intermediate (E)-3-(3,5-bis (benzyloxy)-4-isopropyl styryl) furan (8a)

The above crude product was dissolved in tetrahydrofuran (5 mL). 4-furfural (78.7 mg, 0.7 mmol, 1.5 eq) and sodium hydride (56.4 mg, 2.35 mmol, 5 eq) were added. The reaction was carried out under nitrogen protection at room temperature for 12 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8a (118 mg), which was a yellow oily liquid with a yield of 87%. ¹H NMR (400 MHz, CDCl₃) δ 7.53 (s, 1H), 7.47 (d, J=7.2 Hz, 4H), 7.41 (d, J=6.8 Hz, 4H), 7.39 (d, J=1.2 Hz, 1H), 7.35 (dd, J=6.7, 2.0 Hz, 2H), 7.26 (s, 2H), 6.88 (d, J=16.2 Hz, 1H), 6.75 (s, 2H), 6.64 (d, J=16.2 Hz, 1H), 5.11 (s, 4H), 3.72 (p, J=7.1 Hz, 1H), 1.32 (d, J=7.1 Hz, 6H); GC-MS=424.

### Step 3: Benzyl Functional Groups were Removed to Prepare (E)-5-(2-(furan-3-yl) vinyl)-2-cumene-1,3-diol (9a)

118 mg of compound 8a (0.26 mmol) was weighed in a flask. 261 mg (2.1 mmol, 8 eq) of N,N-dimethylaniline and 177 mg (1 mmol, 4 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9a (57.9 mg), which was a yellow solid with a melting point of 76° C. and a yield of 84.0%, ¹H NMR (400 MHz, MeOD) δ 7.59 (s, 1H), 7.47 (d, J=1.8 Hz, 1H), 6.86 (d, J=16.2 Hz, 1H), 6.71 (d, J=1.9 Hz, 1H), 6.65 (d, J=16.2 Hz, 1H), 6.41 (s, 2H), 3.49 (p, J=7.0 Hz, 1H), 1.31 (d, J=7.1 Hz, 6H); GC-MS=244.

### Embodiment 3

The present embodiment 3 provides a preparation method for the five-membered heterocycle substituted styrene derivative, with a synthetic route as follows:

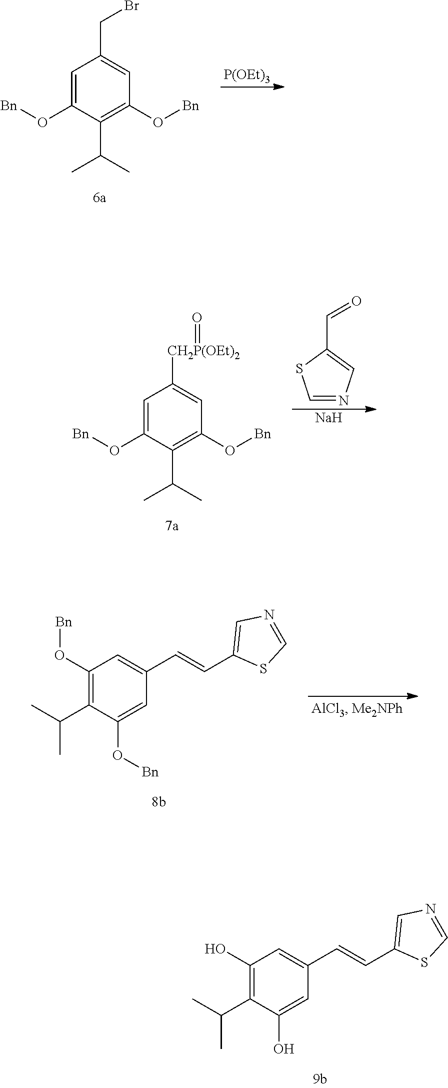

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6a (200 mg, 0.47 mmol, 1 eq) was weighed and added into a 25 mL round-bottomed flask. Triethyl phosphite (5 mL) was added. Tetrabutylammonium bromide (15 mg, 0.047 mmol, 0.1 eq) was added while stirring, and heated to 130° C. The reaction was carried out under nitrogen protection for 6 h. Under heating at 80° C., the excess triethyl phosphite was removed under reduced pressure to obtain a crude product 7a.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-5-(3,5-bis(benzyloxy)-4-isopropyl styryl) thiazole (8b)

The above crude product was dissolved in tetrahydrofuran (5 mL). Thiazole-5-formaldehyde (64 mg, 0.56 mmol, 1.2 eq) and sodium hydride (56.4 mg, 2.35 mmol, 5 eq) were added. Under nitrogen protection, the reaction was carried out at room temperature for 12 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8b (141 mg), which was a yellow oily liquid with a yield of 71%, ¹H NMR (400 MHz, CDCl₃) δ 8.65 (s, 1H), 7.84 (s, 1H), 7.51-7.45 (m, 4H), 7.45-7.38 (m, 4H), 7.37-7.32 (m, 2H), 7.15 (d, J=16.0 Hz, 1H), 6.85 (d, J=16.0 Hz, 1H), 6.74 (s, 2H), 5.12 (s, 4H), 3.73 (h, J=7.1 Hz, 1H), 1.33 (d, J=7.1 Hz, 6H); GC-MS=441.

### Step 3: Benzyl Functional Groups were Removed to Prepare (E)-2-isopropyl-5-(2-(thiazole-5-yl) vinyl)benzene-1,3-diol (9b)

141 mg of compound 8b (0.35 mmol) was weighed in a flask. 324 mg of (2.6 mmol, 8 eq) N,N-dimethylaniline and 221 mg of (1.3 mmol, 4 eq) aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9b (77.1 mg), which was a yellow solid with a melting point of 75° C. and a yield of 84.0%, ¹H NMR (400 MHz, MeOD) δ 8.84 (s, 1H), 7.85 (s, 1H), 7.19 (d, J=16.0 Hz, 1H), 6.79 (d, J=16.0 Hz, 1H), 6.44 (s, 2H), 3.51 (p, J=7.1 Hz, 1H), 1.31 (d, J=7.1 Hz, 7H); GC-MS=261.

### Embodiment 4

The present embodiment 4 provides a preparation method for the five-membered heterocycle substituted styrene derivative, with a synthetic route as follows:

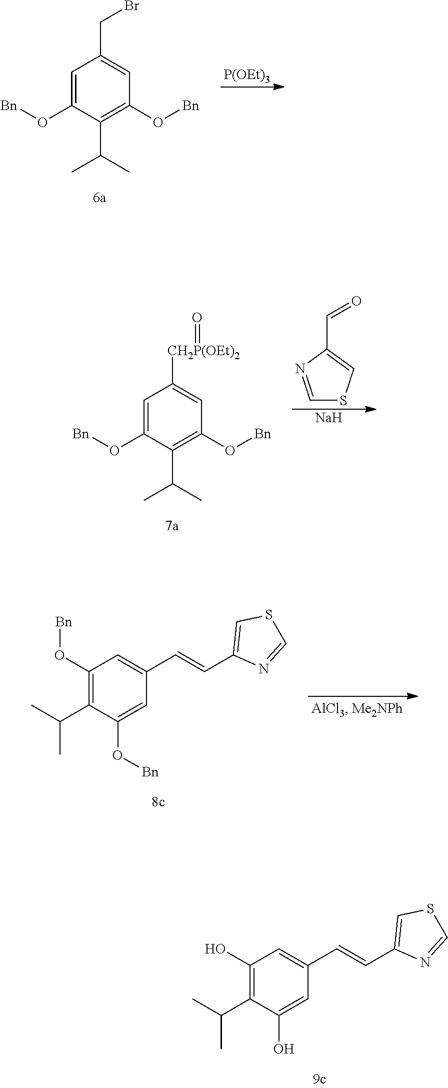

The preparation method includes steps:

### Step 1: a Substitution Reaction of the Styrene Derivative Intermediate and Triethyl Phosphite was Carried Out to Obtain the Styrene Derivative with the Ethyl Phosphite Group

The compound 6a (200 mg, 0.47 mmol, 1 eq) was weighed and added into a 25 mL round-bottomed flask. Triethyl phosphite (5 mL) was added. Tetrabutylammonium bromide (15 mg, 0.047 mmol, 0.1 eq) was added while stirring, and heated to 130° C. The reaction was carried out under nitrogen protection for 6 h. The excess triethyl phosphite was removed under reduced pressure at 80° C. to obtain a crude product 7a.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-4-(3,5-bis(benzyloxy)-4-isopropyl styryl) thiazole (8c)

The above crude product was dissolved in tetrahydrofuran (5 mL). Thiazole-4-formaldehyde (64 mg, 0.56 mmol, 1.2 eq) and sodium hydride (56.4 mg, 2.35 mmol, 5 eq) were added. Under nitrogen protection under TLC monitoring, the reaction was carried out at room temperature for 12 h. After the completion of the reaction, the reaction solution was dissolved in ethyl acetate and washed with water. The organic phase was dried on MgSO₄, and concentrated under reduced pressure. A silica gel column was used for the separation of product 8c (147 mg), which was a yellow oily liquid with a yield of 71%. ¹H NMR (400 MHz, CDCl₃) δ 8.82 (d, J=1.9 Hz, 1H), 7.47 (d, J=7.6 Hz, 4H), 7.41 (d, J=15.9 Hz, 1H), 7.41 (t, J=7.4 Hz, 4H), 7.34 (d, J=7.2 Hz, 2H), 7.21 (d, J=2.0 Hz, 1H), 7.07 (d, J=15.9 Hz, 1H), 6.80 (s, 2H), 5.12 (s, 4H), 3.74 (p, J=7.1 Hz, 1H), 1.32 (d, J=7.1 Hz, 6H); GC-MS=441.

### Step 3: Benzyl Functional Groups were Removed to Prepare (E)-2-isopropyl-5-(2-(thiazole-4-yl) vinyl)benzene-1,3-diol (9c)

147 mg of compound 8c (0.36 mmol) was weighed in the flask. 324 mg of (2.6 mmol, 8 eq) N,N-dimethylaniline and 221 mg of (1.3 mmol, 4 eq) aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9c (76.9 mg), which was a white solid with a melting point of 76° C. and a yield of 84.0%, ¹H NMR (400 MHz, MeOD) δ 9.00 (d, J=2.0 Hz, 1H), 7.48 (d, J=2.0 Hz, 1H), 7.23 (d, J=16.1 Hz, 1H), 7.06 (d, J=16.1 Hz, 1H), 6.48 (s, 2H), 3.51 (p, J=7.1 Hz, 1H), 1.32 (d, J=7.1 Hz, 6H); GC-MS=261.

### Embodiment 5

The present embodiment 5 provides a preparation method for the five-membered heterocycle substituted styrene derivative, with a synthetic route as follows:

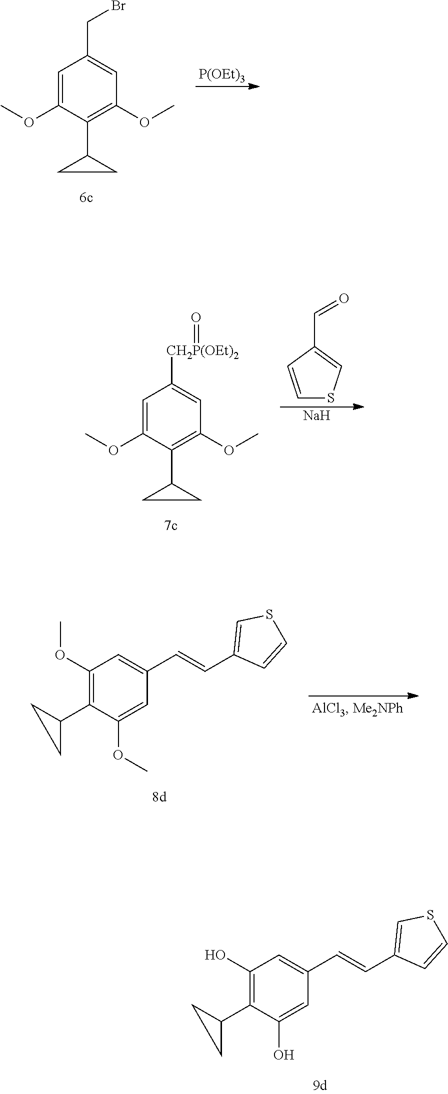

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6c (200 mg, 0.73 mmol, 1 eq) was weighed and added into a 25 mL round-bottomed flask. Triethyl phosphite (5 mL) was added. Tetrabutylammonium bromide (23.6 mg, 0.07 mmol, 0.1 eq) was added while stirring, and heated to 130° C. to react under nitrogen protection for 6 h. Under heating at 80° C., the excess triethyl phosphite was removed under reduced pressure to obtain a crude product 7c.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-cyclopropyl-3,5-dimethoxy styryl) thiophene (8d)

The compound 7c, as raw material was dissolved in tetrahydrofuran (5 mL). 3-thenaldehyde (110 mg, 0.85 mmol, 1.2 eq) and sodium hydride (88.4 mg, 3.51 mmol, 5 eq) were added. Under nitrogen protection, a reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8d (179 mg), which was a yellow solid with a yield of 85%, ¹H NMR (400 MHz, DMSO) δ 7.55-7.46 (m, 2H), 7.42 (dd, J=4.9, 1.4 Hz, 1H), 7.22 (d, J=16.4 Hz, 1H), 6.98 (d, J=16.3 Hz, 1H), 6.73 (s, 2H), 3.74 (s, 6H), 1.80 (tt, J=8.8, 5.7 Hz, 1H), 0.94-0.84 (m, 2H), 0.75-0.64 (m, 2H); GC-MS=286.

### Step 3: Benzyl Functional Groups were Removed to Prepare (E)-2-cyclopropyl-5-(2-(thiophene-3-yl) vinyl)benzene-1,3-diol (9d)

179 mg of compound 8d was weighed in a flask. 610 mg (5 mmol, 8 eq) of N,N-dimethylaniline and 415 mg (2.5 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9d (106.9 mg), which was a yellow solid with a melting point of 96° C. and a yield of 56%, ¹H NMR (400 MHz, MeOD) δ 7.37 (qd, J=5.1, 2.1 Hz, 2H), 7.32 (dd, J=2.8, 1.4 Hz, 1H), 7.02 (d, J=16.3 Hz, 1H), 6.80 (d, J=16.2 Hz, 1H), 6.46 (s, 2H), 1.68 (tt, J=8.3, 6.2 Hz, 1H), 0.89-0.79 (m, 4H); GC-MS=258.

### Embodiment 6

The present embodiment 6 provides a preparation method for the five-membered heterocycle substituted styrene derivative, with a synthetic route as follows:

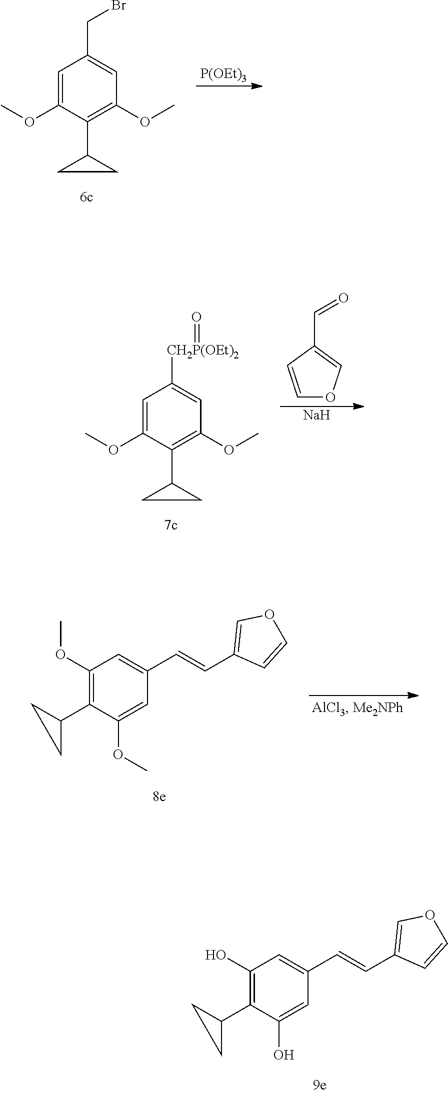

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6c (200 mg, 0.73 mmol, 1 eq) was weighed and added into a 25 mL round-bottomed flask. Triethyl phosphite (5 mL) was added. Tetrabutylammonium bromide (23.6 mg, 0.07 mmol, 0.1 eq) was added while stirring, and heated to 130° C. The reaction was carried out for 6 h under nitrogen protection. Under heating at 80° C., the excess triethyl phosphite was removed under reduced pressure to obtain a crude product 7c.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-cyclopropyl-3,5-dimethoxy styryl) furan (8e)

The compound 7c, as raw material was dissolved in tetrahydrofuran (5 mL). 3-furfural (83 mg, 0.85 mmol, 1.2 eq) and sodium hydride (88.4 mg, 3.51 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8e (147 mg), which was a yellow solid with a yield of 85%.

### Step 3: Benzyl Functional Groups were Removed to Prepare (E)-2-cyclopropyl-5-(2-(furan-3-yl) vinyl)benzene-1,3-diol (9e)

147 mg of compound 8e was weighed in a flask. 530 mg (4.2 mmol, 8 eq) of N,N-dimethylaniline and 355 mg (2.18 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9e (96.9 mg), which was a yellow solid with a melting point of 87° C. and a yield of 71%, ¹H NMR (400 MHz, CDCl₃) δ7.51 (s, 1H), 7.40 (d, J=1.8 Hz, 1H), 7.26 (s, 1H), 6.90 (d, J=16.1 Hz, 1H), 6.70-6.61 (m, 2H), 6.58 (s, 2H), 5.58 (s, 2H), 1.20-1.11 (m, 2H), 0.69 (td, J=5.6, 3.7 Hz, 2H); GC-MS=242.

### Embodiment 7

The present embodiment 7 provides a preparation method for the five-membered heterocycle substituted styrene derivative, with a synthetic route as follows:

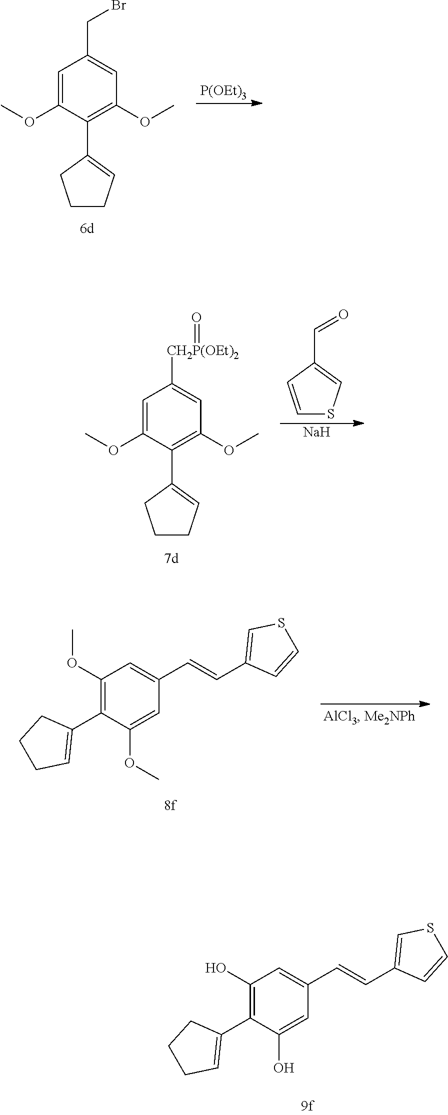

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6d (200 mg, 0.67 mmol, 1 eq) was weighed and added into a 25 mL round-bottomed flask. Triethyl phosphite (5 mL) was added. Tetrabutylammonium bromide (21.6 mg, 0.067 mmol, 0.1 eq) was added while stirring, and heated to 130° C. to react for 6 h under nitrogen protection. Under heating at 80° C., the excess triethyl phosphite was removed under reduced pressure to obtain a crude product 7d.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-(cyclopent-1-ene-1-yl)-3,5-dimethoxy styryl)thiophene (8f)

The compound 7d, as raw material was dissolved in tetrahydrofuran (5 mL). 3-thenaldehyde (93 mg, 0.8 mmol, 1.2 eq) and sodium hydride (85.4 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8f (167 mg), which was a yellow solid with a yield of 85%, 1H NMR (400 MHz, CDCl₃) δ 7.27 (d, J=2.6 Hz, 1H), 7.11 (d, J=16.2 Hz, 1H), 6.92 (d, J=16.2 Hz, 1H), 6.68 (s, 2H), 5.86-5.80 (m, 1H), 3.83 (d, J=2.4 Hz, 6H), 2.66 (ddt, J=8.1, 5.8, 2.4 Hz, 2H), 2.54 (ddt, J=7.7, 5.0, 2.4 Hz, 2H), 1.98 (q, J=7.4 Hz, 2H); GC-MS=312.

### Step 3: Benzyl Functional Groups were Removed to Prepare (E)-2-(cyclopent-1-ene-1-yl)-5-(2-(thiophene-3-yl) vinyl)benzene-1,3-diol (9f)

167 mg of compound 8f was weighed in a flask. 523 mg (4.2 mmol, 8 eq) of N,N-dimethylaniline and 355 mg (2.1 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel columns was used for the separation of product 9f (112.9 mg), which was a yellow solid with a melting point of 89° C. and a yield of 71%, ¹H NMR (400 MHz, CDCl₃) δ 7.32 (t, J=2.0 Hz, 2H), 7.25 (s, 1H), 7.07 (d, J=16.2 Hz, 1H), 6.80 (d, J=16.2 Hz, 1H), 6.61 (s, 2H), 6.01 (p, J=2.1 Hz, 1H), 5.17 (d, J=1.6 Hz, 2H), 2.69 (ddt, J=7.7, 4.8, 2.3 Hz, 2H), 2.61 (tq, J=7.7, 2.5 Hz, 2H), 2.14-2.02 (m, 2H); GC-MS=284.

### Embodiment 8

The present embodiment 8 provides a preparation method for the five-membered heterocycle substituted styrene derivative, with a synthetic route as follows:

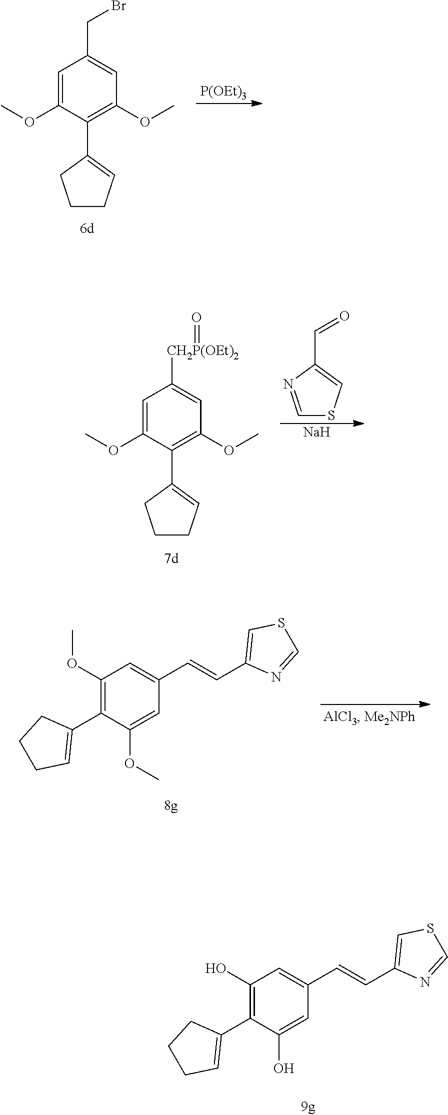

### Step 1: A Substitution Reaction of the Styrene Derivative Intermediate and Triethyl Phosphite was Carried Out to Obtain the Styrene Derivative with the Ethyl Phosphite Group

The compound 6d (200 mg, 0.67 mmol, 1 eq) was weighed and added into a 25 mL round-bottomed flask. Triethyl phosphite (5 mL) was added. Tetrabutylammonium bromide (21.6 mg, 0.067 mmol, 0.1 eq) was added while stirring, and heated to 130° C. to react for 6 h under nitrogen protection. Under heating at 80° C., the excess triethyl phosphite was removed under reduced pressure to obtain a crude product 7d.

### Step 2: The Styrene Derivative with the Ethyl Phosphite Group Undergoes a Substitution Reaction with the Five-Membered Heterocycle to Prepare a Five-Membered Heterocycle Substituted Intermediate Styrene Derivative (E)-4-(4-(cyclopent-1-ene-1-yl)-3,5-dimethoxy styryl) thiazole (8g)

The compound 7d, as raw material was dissolved in tetrahydrofuran (5 mL). Sodium hydride (85.4 mg, 3.50 mmol, 5 eq) was added. After 30 min, thiazole-4-formaldehyde (114 mg, 1.01 mmol, 1.5 eq) was added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8g (196 mg), which was a yellow solid with a yield of 93%, ¹H NMR (400 MHz, CDCl₃) δ8.66 (s, 1H), 7.86 (s, 1H), 7.23 (d, J=16.0 Hz, 1H), 6.89 (d, J=16.0 Hz, 1H), 6.67 (s, 2H), 5.86-5.81 (m, 1H), 3.84 (s, 6H), 2.65 (td, J=7.6, 2.2 Hz, 2H), 2.54 (td, J=8.0, 7.6, 2.4 Hz, 2H), 2.01 (dt, J=14.8, 7.4 Hz, 2H); GC-MS=313.

### Step 3: Benzyl Functional Groups were Removed to Prepare (E)-2-(cyclopent-1-ene-1-yl)-5-(2-(thiazole-4-yl) vinyl)benzene-1,3-diol (9g)

197 mg of compound 8g was weighed in a flask. 523 mg (4.2 mmol, 8 eq) of N,N-dimethylaniline and 355 mg (2.1 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel columns was used for the separation of product 9g (112.9 mg), which was a yellow solid with a melting point of 89° C. and a yield of 71%, ¹H NMR (400 MHz, MeOD) δ 9.00 (d, J=2.0 Hz, 1H), 7.51 (d, J=2.0 Hz, 1H), 7.26 (d, J=16.1 Hz, 1H), 7.12 (d, J=16.1 Hz, 1H), 6.56 (s, 2H), 5.80 (p, J=2.2 Hz, 1H), 2.69 (tq, J=7.0, 2.2 Hz, 2H), 2.53 (tq, J=7.4, 2.5 Hz, 2H), 2.00 (p, J=7.5 Hz, 2H); GC-MS=285.

### Embodiment 9

The present embodiment 9 provides a preparation method for the five-membered heterocycle substituted styrene derivative, with a synthetic route as follows:

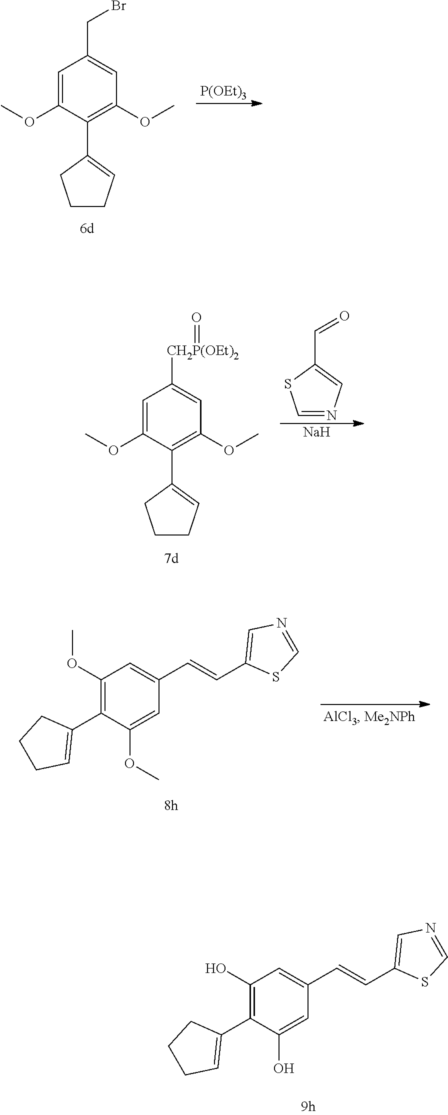

The preparation method includes steps:

### Step 1: a Substitution Reaction of the Styrene Derivative Intermediate and Triethyl Phosphite was Carried Out to Obtain the Styrene Derivative with the Ethyl Phosphite Group

The compound 6d (200 mg, 0.67 mmol, 1 eq) was prepared, weighed and added into a 25 mL round-bottomed flask. Triethyl phosphite (5 mL) was added. Tetrabutylammonium bromide (21.6 mg, 0.067 mmol, 0.1 eq) was added while stirring and heated to 130° C. to react for 6 h under nitrogen protection. Under heating at 80° C., the excess triethyl phosphite was removed under reduced pressure to obtain a crude product 7d.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-5-(4-(cyclopent-1-ene-1-yl)-3,5-dimethoxy styryl) thiazole (8h)

The compound 7d, as raw material was dissolved in tetrahydrofuran (5 mL), Sodium hydride (85.4 mg, 3.50 mmol, 5 eq) was added. After 30 min, thiazole-5-formaldehyde (114 mg, 1.01 mmol, 1.5 eq) was added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8h (176 mg), which was a yellow solid with a yield of 83%, ¹H NMR (400 MHz, CDCl₃) δ 8.66 (s, 1H), 7.86 (s, 1H), 7.23 (d, J=16.0 Hz, 1H), 6.89 (d, J=16.0 Hz, 1H), 6.67 (s, 2H), 5.86-5.81 (m, 1H), 3.84 (s, 6H), 2.65 (td, J=7.6, 2.2 Hz, 2H), 2.54 (td, J=8.0, 7.6, 2.4 Hz, 2H), 2.01 (dt, J=14.8, 7.4 Hz, 2H); GC-MS=313.

### Step 3: Benzyl Functional Groups were Removed to Prepare (E)-2-(cyclopent-1-ene-1-yl)-5-(2-(thiazole-5-yl) vinyl)benzene-1,3-diol (9h)

176 mg of compound 8 h was weighed in a flask. 548 mg (4.5 mmol, 8 eq) of N,N-dimethylaniline and 375 mg (2.3 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9h (112 mg), which was a yellow solid with a melting point of 89° C. and a yield of 70%. ¹H NMR (400 MHz, MeOD) δ 8.85 (s, 1H), 7.88 (s, 1H), 7.26 (d, J=16.0 Hz, 1H), 6.83 (d, J=16.0 Hz, 1H), 6.53 (s, 2H), 5.80 (t, J=2.2 Hz, 1H), 2.68 (tq, J=7.5, 2.2 Hz, 2H), 2.53 (tq, J=7.3, 2.4 Hz, 2H), 2.06-1.94 (m, 2H); GC MS=285.

### Embodiment 10

The present embodiment 10 provides a preparation method for the five-membered heterocycle substituted styrene derivative, with a synthetic route as follows:

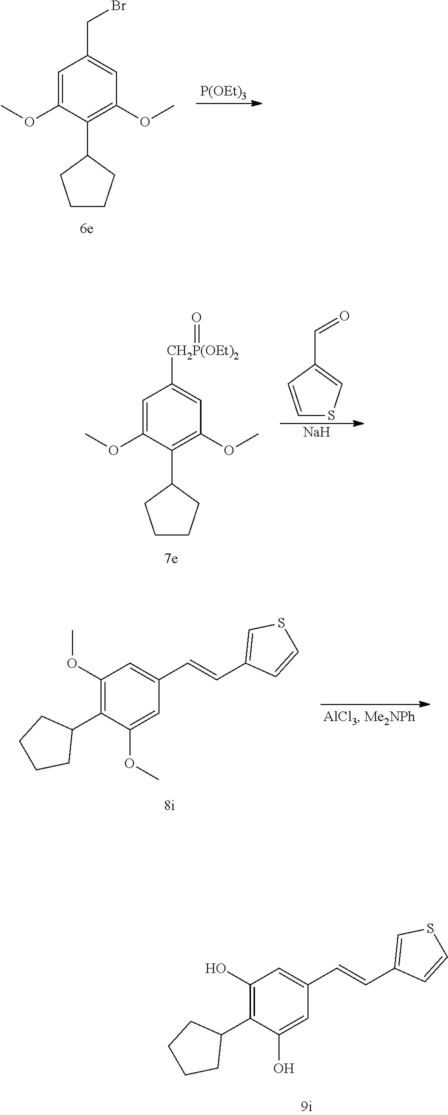

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6e (200 mg, 0.67 mmol, 1 eq) was weighed and added into a 25 mL round-bottomed flask. Triethyl phosphite (5 mL) was added. Tetrabutylammonium bromide (21.6 mg, 0.067 mmol, 0.1 eq) was added while stirring, and heated to 130° C. to react for 6 h under nitrogen protection. Under heating at 80° C., the excess triethyl phosphite was removed under reduced pressure to obtain a crude product 7e.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-cyclopentyl-3,5-dimethoxy styryl) thiophene (8i)

The compound 7e, as raw material was dissolved in tetrahydrofuran (5 mL). 3-thenaldehyde (93 mg, 0.8 mmol, 1.2 eq) and sodium hydride (85.4 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8i (178 mg), which was a yellow solid with a yield of 85%, 1H NMR (400 MHz, CDCl₃) δ 7.33 (qd, J=5.2, 2.1 Hz, 2H), 7.25 (s, 1H), 7.07 (d, J=16.2 Hz, 1H), 6.90 (d, J=16.2 Hz, 1H), 6.66 (s, 2H), 3.85 (s, 6H), 3.67-3.61 (m, 1H), 1.93-1.70 (m, 6H), 1.61 (d, J=6.2 Hz, 2H); GC-MS=314.

### Step 3: Benzyl Functional Groups were Removed to Prepare (E)-2-cyclopentyl-5-(2-(thiophene-3-yl) vinyl)benzene-1,3-diol (9i)

178 mg of compound 8i was weighed in a flask. 533 mg (4.3 mmol, 8 eq) of N,N-dimethylaniline and 360 mg (2.2 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9i (119 mg), which was a yellow solid with a melting point of 89° C. and a yield of 72%, ¹H NMR (400 MHz, CDCl₃) δ 7.31 (d, J=2.1 Hz, 2H), 7.24 (s, 1H), 7.00 (d, J=16.2 Hz, 1H), 6.75 (d, J=16.2 Hz, 1H), 6.48 (s, 2H), 4.78 (s, 2H), 3.44 (p, J=9.0 Hz, 1H), 2.06-1.93 (m, 2H), 1.88 (tt, J=10.6, 6.8 Hz, 4H), 1.67 (s, 2H); GC-MS=286.

### Embodiment 11

The present embodiment 11 provides a preparation method for the five-membered heterocycle substituted styrene derivative, with a synthetic route as follows:

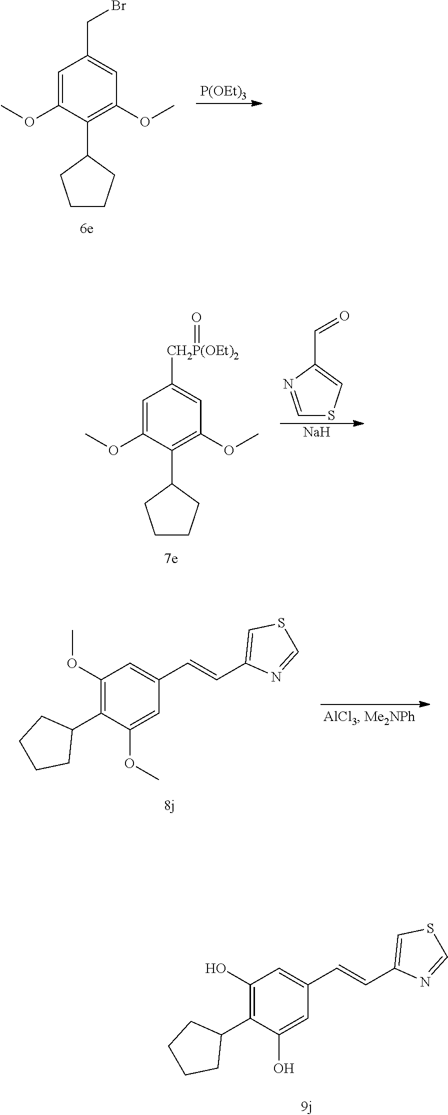

The preparation method includes steps:

### Step 1: a Substitution Reaction of the Styrene Derivative Intermediate and Triethyl Phosphite was Carried Out to Obtain the Styrene Derivative with the Ethyl Phosphite Group

The compound 6e (200 mg, 0.67 mmol, 1 eq) was weighed and added into a 25 mL round-bottomed flask. Triethyl phosphite (5 mL) was added. Tetrabutylammonium bromide (21.6 mg, 0.067 mmol, 0.1 eq) was added while stirring and heated to 130° C. The reaction was carried out for 6 h under nitrogen protection. Under heating at 80° C., the excess triethyl phosphite was removed under reduced pressure to obtain a crude product 7e.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-4-(4-cyclopentyl-3,5-dimethoxy styryl) thiazole (8j)

The compound 7e, as raw material was dissolved in tetrahydrofuran (5 mL). Thiazole-4-formaldehyde (113 mg, 1.0 mmol, 1.5 eq) and sodium hydride (85.4 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8j (193 mg), which was a yellow solid with a yield of 92%, ¹H NMR (400 MHz, CDCl₃) δ 8.82 (d, J=1.9 Hz, 1H), 7.46 (d, J=15.9 Hz, 1H), 7.21 (d, J=2.0 Hz, 1H), 7.12 (d, J=16.0 Hz, 1H), 6.72 (s, 2H), 3.84 (s, 6H), 1.98-1.68 (m, 8H); GC-MS=315,

### Step 3: Benzyl Functional Groups were Removed to Prepare (E)-2-cyclopentyl-5-(2-(thiazole-4-yl) vinyl)benzene-1,3-diol (9j)

193 mg of compound 8j was weighed in a flask. 535 mg (4.3 mmol, 8 eq) of N,N-dimethylaniline and 362 mg (2.2 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9j (112 mg), which was a yellow solid with a melting point of 73° C. and a yield of 71%, ¹H NMR (400 MHz, MeOD) δ 8.99 (d, J=2.0 Hz, 1H), 7.47 (d, J=2.0 Hz, 1H), 7.23 (d, J=16.0 Hz, 1H), 7.06 (d, J=16.1 Hz, 1H), 6.49 (s, 2H), 3.63-3.49 (m, 1H), 2.18-2.05 (m, 2H), 1.87 (qd, J=7.9, 6.5, 3.4 Hz, 2H), 1.79-1.68 (m, 2H), 1.63 (qd, J=7.4, 6.9, 3.3 Hz, 2H); GC-MS=287.

### Embodiment 12

The present embodiment 12 provides a preparation method for the five-membered heterocycle substituted styrene derivative, with a synthetic route as follows:

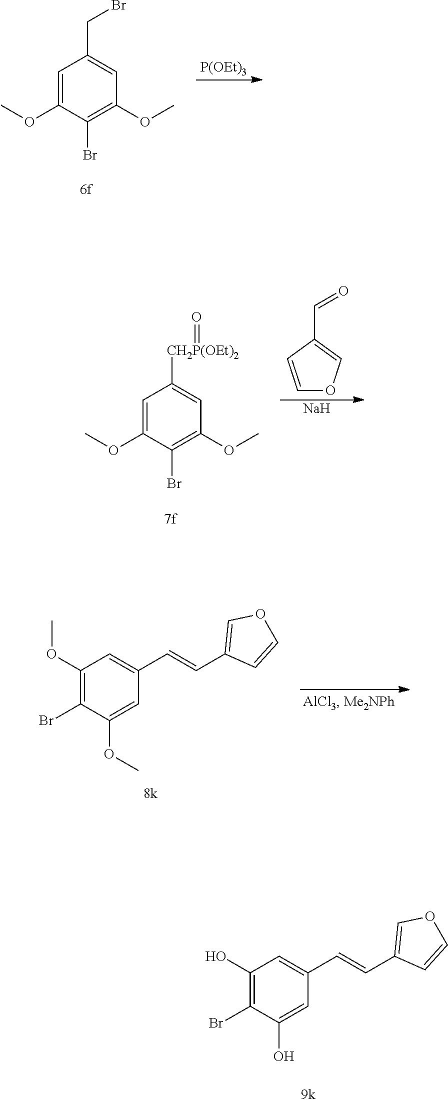

The preparation method includes steps:

### Step 1: The Substitution Reaction of the Styrene Derivative Intermediate and Triethyl Phosphite was Carried Out to Obtain the Styrene Derivative with the Ethyl Phosphite Group

The compound 6f (200 mg, 0.64 mmol, 1 eq) was weighed and added into a 25 mL round-bottomed flask. Triethyl phosphite (5 mL) was added. Tetrabutylammonium bromide (21.6 mg, 0.06 mmol, 0.1 eq) was added while stirring and heated to 130° C. to react for 6 h under nitrogen protection. Under heating at 80° C., the excess triethyl phosphite was removed under reduced pressure to obtain a crude product 7f.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-bromo-3,5-dimethoxy styryl) furan (8k)

The compound 7f, as raw material was dissolved in tetrahydrofuran (5 mL). 3-furfural (78 mg, 0.8 mmol, 1.2 eq) and sodium hydride (85.4 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8k (167 mg), which was a yellow solid with a yield of 85%, ¹H NMR (400 MHz, CDCl₃) δ 7.59 (s, 1H), 7.46 (s, 1H), 6.88 (d, J=16.2 Hz, 1H), 6.75 (s, 1H), 6.64 (d, J=16.2 Hz, 1H), 6.47 (s, 2H), 5.12 (dq, J=3.0, 1.5 Hz, 1H), 4.67 (dd, J=2.4, 1.1 Hz, 1H), 3.71 (s, 6H), 1.83 (s, J=1.2 Hz, 3H); GC-MS=309.

### Step 3: Benzyl Functional Groups were Removed to Prepare (E)-2-bromo-5-(2-(furan-3-yl) vinyl)benzene-1,3-diol (9k)

167 mg of compound 8k (0.54 mmol) was weighed in a flask. 524 mg (4.6 mmol, 8 eq) of N,N-dimethylaniline and 356 mg (2.1 mmol, 4 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9k (108.1 mg), which was a yellow solid with a melting point of 75° C. and a yield of 66%, ¹H NMR (400 MHz, CDCl₃) δ 7.54 (s, 1H), 7.41 (d, J=1.8 Hz, 1H), 6.94 (d, J=16.1 Hz, 1H), 6.71 (s, 2H), 6.67 (d, 1H), 6.63 (s, 1H), 5.39 (s, 2H); GC-MS=281.

### Embodiment 13

The present embodiment 13 provides a preparation method for the five-membered heterocycle substituted styrene derivative, with a synthetic route as follows:

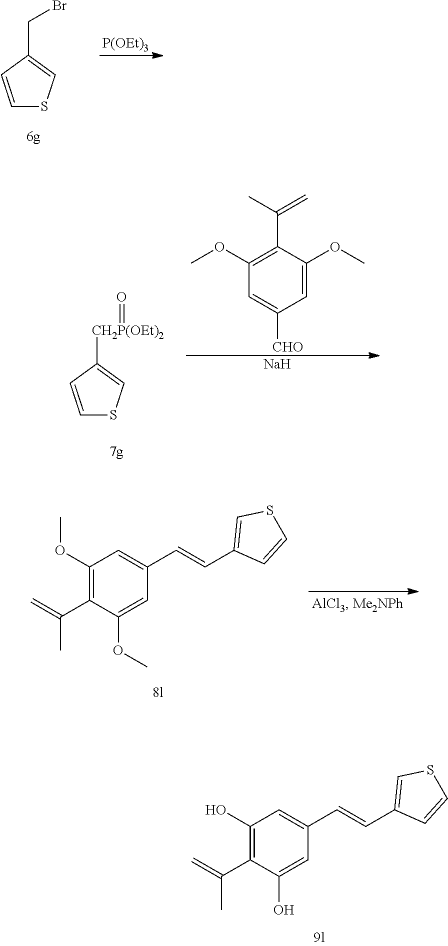

The preparation method includes steps:

### Step 1: A Substitution Reaction of the Styrene Derivative Intermediate and Triethyl Phosphite was Carried Out to Obtain the Styrene Derivative with the Ethyl Phosphite Group

3-(bromomethyl)thiophene (200 mg, 1.12 mmol, 1 eq) was weighed and added into a 25 mL round-bottomed flask. Triethyl phosphite (5 mL) was added. Tetrabutylammonium bromide (35 mg, 0.107 mmol, 0.1 eq) was added while stirring and heated to 130° C. to react for 6 h under nitrogen protection. Under heating at 80° C., the excess triethyl phosphite was removed under reduced pressure to obtain a crude product 7g.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(3,5-dimethoxy-4-(prop-1-ene-2-yl) styryl)thiophene

The compound 7g, as raw material was dissolved in tetrahydrofuran (5 mL). 3,5-dimethoxy-4-(1-methylvinyl)benzaldehyde (280 mg, 1.35 mmol, 1.2 eq) and sodium hydride (135.4 mg, 5.55 mmol, 5 eq) were added to react at room temperature for 12 h under nitrogen protection. After the completion of the reaction under TLC monitoring, the reaction solution was dissolved in ethyl acetate and washed with water. The organic phase was dried on MgSO₄, and concentrated under reduced pressure. A silica gel column was used for the separation of product 81 (274 mg), which was a yellow solid with a yield of 85%, ¹H NMR (400 MHz, DMSO) δ 7.55-7.48 (m, 2H), 7.42 (s, J=4.5, 1.8 Hz, 1H), 7.26 (d, J=16.3 Hz, 1H), 7.01 (d, J=16.5 Hz, 1H), 6.78 (s, 2H), 5.12 (dq, J=3.0, 1.5 Hz, 1H), 4.67 (dd, J=2.4, 1.1 Hz, 1H), 3.71 (s, 6H), 1.83 (s, J=1.2 Hz, 3H); GC-MS=286.

### Step 3: Benzyl Functional Groups were Removed to Prepare (E)-2-(prop-1-ene-2-yl)-5-(2-(thiophene-3-yl) vinyl)benzene-1,3-diol (9l)

274 mg of compound 8l (0.95 mmol) was weighed in a flask. 924 mg (7.6 mmol, 8 eq) of N,N-dimethylaniline and 636 mg (3.8 mmol, 4 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation to obtain a product 91 (128.1 mg), which was a yellow solid with a melting point of 75° C. and a yield of 56.0%, ¹H NMR (400 MHz, MeOD) δ 7.41-7.35 (m, 2H), 7.35-7.31 (m, 1H), 7.05 (d, J=16.3 Hz, 1H), 6.81 (d, J=16.3 Hz, 1H), 6.50 (s, 2H), 5.30 (dt, J=2.9, 1.5 Hz, 1H), 4.92-4.89 (m, 1H), 2.02 (d, J=1.3 Hz, 3H); GC-MS=258.

### Embodiment 14

The present embodiment 14 provides a preparation method for the five-membered heterocycle substituted styrene derivative, with a synthetic route as follows:

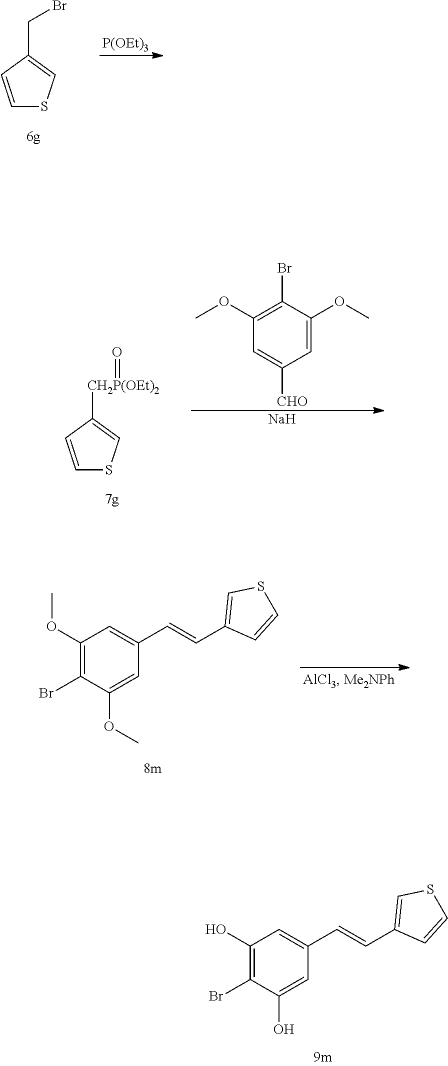

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6g (200 mg, 1.12 mmol, 1 eq) was weighed and added into a 25 mL round-bottomed flask. Triethyl phosphite (5 mL) was added. Tetrabutylammonium bromide (35 mg, 0.107 mmol, 0.1 eq) was added while stirring and heated to 130° C. to react for 6 h under nitrogen protection. Under heating at 80° C., the excess triethyl phosphite was removed under reduced pressure to obtain a crude product 7g.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(4-bromo-3,5-dimethoxy styryl) thiophene (8m)

The compound 7g, as raw material was dissolved in tetrahydrofuran (5 mL). 4-bromo-3,5-dimethoxybenzaldehyde (280 mg, 1.35 mmol, 1.2 eq) and sodium hydride (135.4 mg, 5.55 mmol, 5 eq) were added. The reaction was carried out at room temperature for 12 h under nitrogen protection. After the completion of the reaction under TLC monitoring, the reaction solution was dissolved in ethyl acetate and washed with water. The organic phase was dried on MgSO₄, and concentrated under reduced pressure. A silica gel column were used for the separation of product 8m (274 mg), which was a yellow solid with a yield of 85%.

### Step 3: Benzyl Functional Groups were Removed to Prepare (E)-2-bromo-5-(2-(thiophene-3-yl) vinyl)benzene-1,3-diol (9m)

274 mg of compound 8m (0.95 mmol) was weighed in a flask. 924 mg (7.6 mmol, 8 eq) of N,N-dimethylaniline and 636 mg (3.8 mmol, 4 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9m (148.1 mg), which was a yellow solid with a melting point of 75° C. and a yield of 77%, ¹H NMR (400 MHz, CDCl₃) δ 7.32 (d, J=2.1 Hz, 2H), 7.28 (d, J=2.2 Hz, 1H), 7.09 (d, J=16.2 Hz, 1H), 6.79 (d, J=16.2 Hz, 1H), 6.73 (s, 2H), 5.40 (s, 2H); GC-MS=297.

### Embodiment 15

The present embodiment 15 provides a preparation method for the five-membered heterocycle substituted styrene derivative, with a synthetic route as follows:

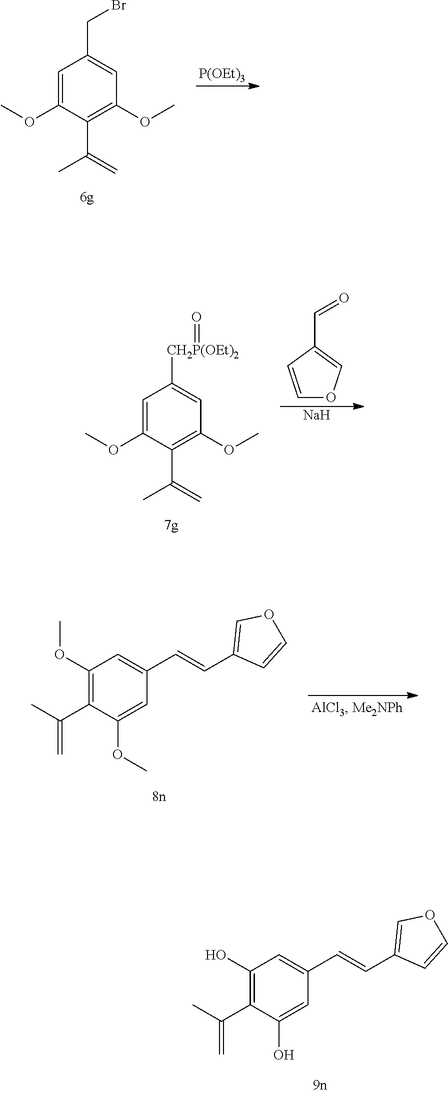

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6g (200 mg, 1.12 mmol, 1 eq) was weighed and added into a 25 mL round-bottomed flask. Triethyl phosphite (5 mL) was added. Tetrabutylammonium bromide (35 mg, 0.107 mmol, 0.1 eq) was added while stirring, and heated to 130° C. to react for 6 h under nitrogen protection. Under heating at 80° C., the excess triethyl phosphite was removed under reduced pressure to obtain a crude product 7g.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-3-(3,5-dimethoxy-4-(prop-1-ene-2-yl) styryl) furan (8n)

The compound 7g, as raw material was dissolved in tetrahydrofuran (5 mL). 3-methylfuran (138 mg, 1.45 mmol, 2 eq) and sodium hydride (69 mg, 2.9 mmol, 5 eq) were added to react at room temperature for 12 h under nitrogen protection. After the completion of the reaction under TLC monitoring, the reaction solution was dissolved in ethyl acetate and washed with water. The organic phase was dried on MgSO₄, and concentrated under reduced pressure. A silica gel column was used for the separation of product 8n (166 mg), which was a yellow solid with a yield of 85%, ¹H NMR (400 MHz, DMSO) δ 7.59 (s, 1H), 7.46 (s, 1H), 7.06 (d, J=16.3 Hz, 1H), 6.95 (s, 1H), 6.82 (d, J=16.3 Hz, 1H), 6.78 (s, 2H), 5.12 (dq, J=3.0, 1.5 Hz, 1H), 4.67 (dd, J=2.4, 1.1 Hz, 1H), 3.71 (s, 6H), 1.83 (s, J=1.2 Hz, 3H); GC-MS=270.

### Step 3: Benzyl Functional Groups were Removed to Prepare (E)-5-(2-(furan-3-yl) vinyl)-2-(prop-1-ene-2-yl)benzene-1,3-diol (9n)

166 mg of compound 8n (0.95 mmol) was weighed in a flask. 724 mg (5.8 mmol, 8 eq) of N,N-dimethylaniline and 736 mg (4.8 mmol, 6 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation to obtain a product 9n (148 mg), which was a yellow solid with a melting point of 72° C. and a yield of 67%, ¹H NMR (400 MHz, MeOD) δ 7.60 (s, 1H), 7.47 (s, 1H), 6.91 (d, J=16.2 Hz, 1H), 6.72 (s, 1H), 6.68 (d, J=16.2 Hz, 1H), 6.48 (s, 2H), 5.31 (q, J=1.8 Hz, 1H), 4.89 (q, J=1.8 Hz, 1H), 2.03 (d, J=1.3 Hz, 3H); GC-MS=242.

### Embodiment 16

The present embodiment 16 provides a preparation method for the five-membered heterocycle substituted styrene derivative, with a synthetic route as follows:

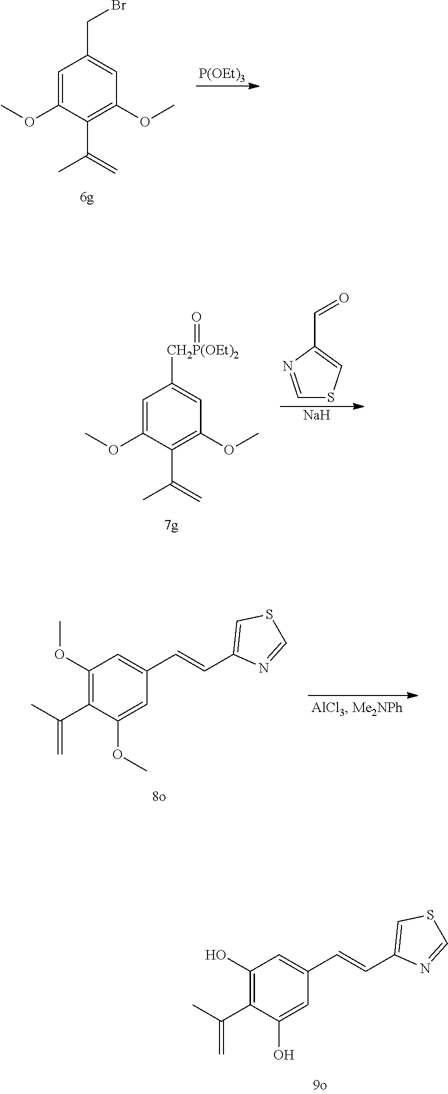

The preparation method includes steps:

### Step 1: a Substitution Reaction of the Styrene Derivative Intermediate and Triethyl Phosphite was Carried Out to Obtain the Styrene Derivative with the Ethyl Phosphite Group

The compound 6g (200 mg, 0.73 mmol, 1 eq) was weighed and added into a 25 mL round-bottomed flask. Triethyl phosphite (5 mL) was added. Tetrabutylammonium bromide (31.6 mg, 0.07 mmol, 0.1 eq) was added while stirring, and heated to 130° C. to react for 6 h under nitrogen protection. Under heating at 80° C., the excess triethyl phosphite was removed under reduced pressure to obtain a crude product 7g.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-4-(3,5-dimethoxy-4-(prop-1-ene-2-yl) styryl) thiazole (8o)

The compound 7g, as raw material was dissolved in tetrahydrofuran (5 mL). Thiazole-4-formaldehyde (125 mg, 1.1 mmol, 1.5 eq) and sodium hydride (88.7 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel column was used for the separation of product 8o (178 mg), which was a yellow solid with a yield of 85%. ¹H NMR (400 MHz, CDCl₃) δ 8.83 (d, J=2.1 Hz, 1H), 7.48 (d, J=16.0 Hz, 1H), 7.24 (d, J=1.9 Hz, 1H), 7.15 (d, J=15.9 Hz, 1H), 6.75 (s, 2H), 5.37-5.31 (m, 1H), 4.89 (dd, J=2.2, 1.1 Hz, 1H), 3.85 (s, 6H), 3.82 (s, 3H); GC-MS=287.

### Step 3: Benzyl Functional Groups were Removed to Prepare (E)-2-(prop-1-ene-2-yl)-5-(2-(thiazole-4-yl) vinyl)benzene-1,3-diol (9o)

178 mg of compound 8o was weighed in a flask. 533 mg (4.3 mmol, 8 eq) of N,N-dimethylaniline and 360 mg (2.2 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9o (119 mg), which was a yellow solid with a melting point of 89° C. and a yield of 72%. ¹H NMR (400 MHz, MeOD) δ 9.00 (d, J=2.0 Hz, 1H), 7.51 (d, J=2.0 Hz, 1H), 7.26 (d, J=16.1 Hz, 1H), 7.11 (d, J=16.1 Hz, 1H), 6.56 (s, 2H), 5.31 (dt, J=3.1, 1.5 Hz, 1H), 4.91 (dt, J=3.1, 1.5 Hz, 1H), 2.03 (d, J=1.4 Hz, 3H); GC-MS=259.

### Embodiment 17

The present embodiment 17 provides a preparation method for the five-membered heterocycle substituted styrene derivative, with a synthetic route as follows:

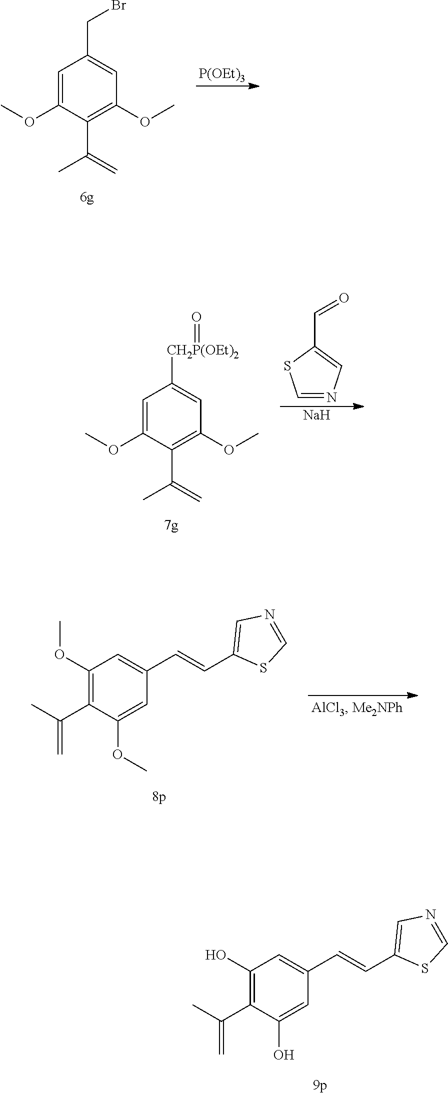

The preparation method includes steps:

### Step 1: A Substitution Reaction of the Styrene Derivative Intermediate and Triethyl Phosphite was Carried Out to Obtain the Styrene Derivative with the Ethyl Phosphite Group

The compound 6g (200 mg, 0.73 mmol, 1 eq) was weighed and added into a 25 mL round-bottomed flask. Triethyl phosphite (5 mL) was added. Tetrabutylammonium bromide (31.6 mg, 0.07 mmol, 0.1 eq) was added while stirring, and heated to 130° C. to react for 6 h under nitrogen protection. Under heating at 80° C., the excess triethyl phosphite was removed under reduced pressure to obtain a crude product 7g.

### Step 2: The Styrene Derivative with the Ethyl Phosphite Group Undergoes a Substitution Reaction with the Five-Membered Heterocycle to Prepare a Five-Membered Heterocycle Substituted Styrene Derivative Intermediate (E)-5-(3,5-dimethoxy-4-(prop-1-ene-2-yl) styryl) thiazole (8p)

The compound 7g, as raw material was dissolved in tetrahydrofuran (5 mL). Thiazole-5-formaldehyde (125 mg, 1.1 mmol, 1.5 eq) and sodium hydride (88.7 mg, 3.5 mmol, 5 eq) were added. Under nitrogen protection, the reaction was carried out at room temperature for 4 h. After the completion of the reaction under TLC monitoring, the reaction solution was dissolved in ethyl acetate and washed with water. The organic phase was dried on MgSO₄, and concentrated under reduced pressure. A silica gel columns were used for the separation of product 8p (171 mg), which was a yellow solid with a yield of 81%, ¹H NMR (400 MHz, CDCl₃) δ 8.69 (s, 1H), 7.89 (s, 1H), 7.25 (d, J=16.0 Hz, 1H), 6.96-6.87 (d, J=16.0 Hz, 1H), 6.70 (s, 2H), 5.36 (t, J=1.9 Hz, 1H), 4.91 (dd, J=2.3, 1.1 Hz, 1H), 3.88 (s, 6H), 2.04 (s, 3H); GC-MS=287.

### Step 3: Benzyl Functional Groups were Removed to Prepare (E)-2-(prop-1-ene-2-yl)-5-(2-(thiazole-5-yl) vinyl)benzene-1,3-diol (9p)

171 mg of compound 8p was weighed into a flask; and 513 mg (4.1 mmol, 8 eq) of N,N-dimethylaniline, and 356 mg (2.1 mmol, 4 eq) of aluminum trichloride were added to react at room temperature for 5 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9p (109 mg), which was a yellow solid with a melting point of 89° C. and a yield of 67%, ¹H NMR (400 MHz, MeOD) δ 8.86 (s, 1H), 7.88 (s, 1H), 7.26 (d, J=16.1 Hz, 1H), 6.84 (d, J=16.1 Hz, 1H), 6.53 (s, 2H), 5.33 (dt, J=2.5, 1.5 Hz, 1H), 4.92 (dt, J=2.4, 1.1 Hz, 1H), 2.04 (t, J=1.2 Hz, 3H); GC-MS=259.

### Embodiment 18

The present embodiment 18 provides a preparation method for the five-membered heterocycle substituted styrene derivative, with a synthetic route as follows:

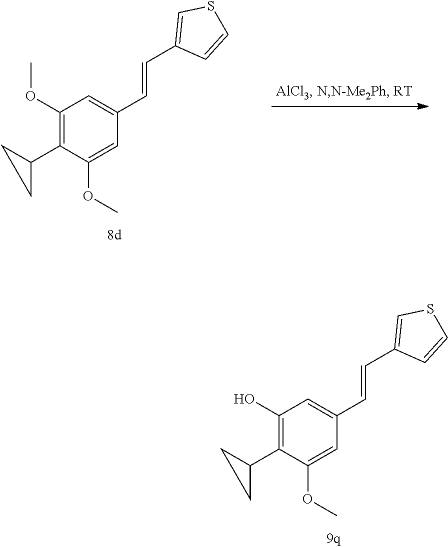 [SMILES: COc1cc(/C=C/c2ccsc2)cc(OC)c1C1CC1 ; COc1cc(/C=C/c2ccsc2)cc(O)c1C1CC1]

The preparation method includes steps:

### Step 1: (E)-2-cyclopropyl-3-methoxy-5-(2-(thiophene-3-yl) vinyl) phenol (9q) was Prepared

150 mg of compound 8d (0.52 mmol) was weighed in a flask. 254 mg (2.06 mmol, 4 eq) of N,N-dimethylaniline and 261 mg (1.5 mmol, 3 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9q (101 mg), which was a yellow solid with a melting point of 75° C. and a yield of 72%, ¹H NMR (400 MHz, CDCl₃) δ 7.36-7.28 (m, 2H), 7.07 (d, J=16.2 Hz, 1H), 6.85 (d, J=16.2 Hz, 1H), 6.68 (d, J=1.6 Hz, 1H), 6.55 (d, J=1.6 Hz, 1H), 5.87 (s, 1H), 3.88 (s, 3H), 1.50 (tt, J=8.1, 5.5 Hz, 1H), 1.08-0.99 (m, 2H), 0.68-0.60 (m, 2H); GC-MS=272.

### Embodiment 19

The present embodiment 19 provides a preparation method for the five-membered heterocycle substituted styrene derivative, with a synthetic route as follows:

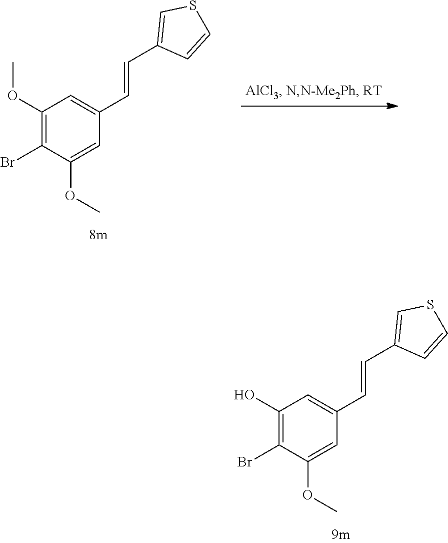 [SMILES: COc1cc(/C=C/c2ccsc2)cc(OC)c1Br ; COc1cc(/C=C/c2ccsc2)cc(O)c1Br]

The preparation method includes steps:

### Step 1: (E)-2-bromo-3-methoxy-5-(2-(thiophene-3-yl) vinyl) phenol (9m) was Prepared

150 mg of compound 8m (0.52 mmol) was weighed in a flask. 225 mg (1.86 mmol, 4 eq) of N,N-dimethylaniline, and 161 mg (0.9 mmol, 2 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9m (107 mg), which was a yellow solid with a melting point of 75° C. and a yield of 75%, ¹H NMR (400 MHz, CDCl₃) δ 7.36-7.31 (m, 2H), 7.29 (d, J=2.2 Hz, 1H), 7.12 (d, J=16.2 Hz, 1H), 6.87 (s, 1H), 6.84 (s, 1H), 6.79 (s, 1H), 6.59 (d, J=1.9 Hz, 1H), 5.63 (s, 1H), 3.94 (s, 3H); GC-MS=311.

### Embodiment 20

The present embodiment 20 provides a preparation method for the five-membered heterocycle substituted styrene derivative, with a synthetic route as follows:

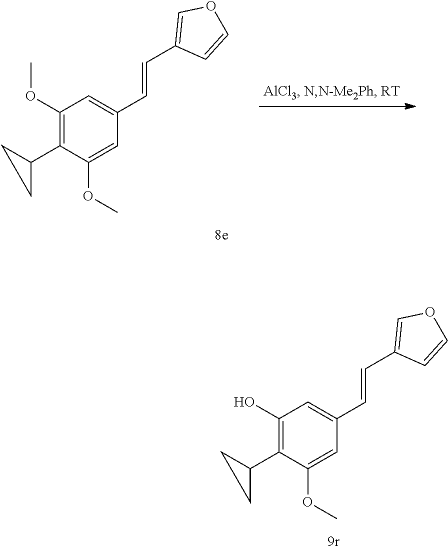 [SMILES: COc1cc(/C=C/c2ccoc2)cc(OC)c1C1CC1 ; COc1cc(/C=C/c2ccoc2)cc(O)c1C1CC1]

The preparation method includes steps:

### Step 1: (E)-5-(2-(furan-3-yl) vinyl)-2-isopropyl-3-methoxyphenol (9r) was Prepared

150 mg of compound 8e (0.52 mmol) was weighed in a flask. 274 mg (2.26 mmol, 4 eq) of N,N-dimethylaniline and 184 mg (1.1 mmol, 2 eq) of aluminum trichloride were added to react at room temperature for 4 h. After the completion of the reaction under TLC monitoring, the mixture was dissolved in ethyl acetate. Hydrochloric acid was added to adjust the pH of aqueous phase to 1-2. The organic phase was dried on MgSO₄, and concentrated under reduced pressure. A silica gel column was used for the separation of product 9r (101 mg), which was a yellow solid with a melting point of 75° C. and a yield of 72%. ¹H NMR (400 MHz, CDCl₃) δ 7.52 (d, J=1.4 Hz, 1H), 7.41 (d, J=1.9 Hz, 1H), 6.88 (d, J=16.1 Hz, 1H), 6.69 (d, J=16.1 Hz, 1H), 6.63 (d, J=1.9 Hz, 1H), 6.56 (d, J=1.5 Hz, 1H), 6.48 (d, J=1.6 Hz, 1H), 4.71 (s, 1H), 3.84 (s, 3H), 3.50 (p, J=7.1 Hz, 1H), 1.32 (d, J=7.1 Hz, 6H); GC-MS=258.

### Test Example 1

In this test example, the styrene derivatives prepared by embodiments 2-20 were tested for their toxicities to mouse macrophages (RAW264.7). The nitric oxide (NO) inhibition assays using lipopolysaccharide (LPS)-induced RAW264.7 cells were also performed to evaluate their anti-inflammatory activities (Test methods refer to Zhou W. et al. Biomed Pharmacother 2020, 131:110696.).

### (1) the Toxicity to Macrophages was Tested by MTT Method:

Mouse macrophages (RAW264.7) were taken, inoculated into 96-well plates (100 L) at a concentration of 3×10⁴/mL and cultured in an incubator of 5% CO₂ at 37° C. for 18 h. After the cells adhered to the wall, the supernate was discarded and compounds of different concentrations were added to continue the culture for 48 h. 0.5 mg/mL of MTT was added. After continued culture for 4 h, 150 OL of DMSO was added to each well, and then shaken on a shaker for 10 min. Absorbance (OD value) at 570 nm was measured by a microplate reader, and IC₅₀ was calculated by Prism software. Results are shown in Table 1.

### (2) Inhibition of LPS-Induced NO Production Activity in Macrophages was Tested (the Test Method Refers to Biomed Pharmacother. 2020 November; 131:110696):

Mouse macrophages (RAW264.7) were taken, inoculated into 96-well plates (100 L) at a concentration of 5×10⁵/mL and cultured in a 5% CO₂ incubator at 37° C. for 18 h. Then, the compound (final concentration: 10 M) and LPS (final concentration: 5 g/mL) were added into a cell culture solution and cultured at 37° C. for 24 h. Then, the supernatant of the cell culture solution was taken and placed into a new 96-well plate by 50 L per well; and then 50 L of solution A and 50 L of solution B of a Beyotime NO kit were added.

After culturing in the incubator at 37° C. for 10 min, the absorbance (OD value) at 540 nm was tested. Results are shown in Table 1.

It can be seen from Table 1 that the compounds synthesized by the present invention are less toxic to macrophages and show high inhibitory activity against nitric oxide. It is shown that these compounds have good anti-inflammatory activity, are new anti-inflammatory compounds and can be used as anti-inflammatory drugs to treat various inflammations.

TABLE 1
Test results of activity of styrene derivatives
prepared in embodiments 2-11

| | | | Inhibitory |
|---|---|---|---|
| | | | activity |
| | | | against |
| | | Toxicity to | nitric oxide |
| | | macrophages | production |
| No | Compound | IC₅₀ (μM) | (%) at 10M |
| 2 | (E)-5-(2-(furan-3-yl)vinyl)-2- | >1 | <50 |
| | isopropyl-1,3-diol | | |
| 3 | (E)-2-isopropyl- | >1 | >50 |
| | 5-(2-(thiazole-5- | | |
| | yl)vinyl)benzene-1,3-diol | | |
| 4 | (E)-2-isopropyl-5-(2-(thiazole-4- | >1 | <50 |
| | yl)vinyl)benzene-1,3-diol | | |
| 5 | (E)-2-cyclopropyl-5-(2-(thiophene-3- | >1 | <50 |
| | yl)vinyl)benzene-1,3-diol | | |
| 6 | (E)-2-cyclopropyl-5-(2-(furan-3- | >1 | <50 |
| | yl)vinyl)benzene-1,3-diol | | |
| 7 | (E)-2-(cyclopent-1-ene-1-yl)-5-(2- | >1 | <50 |
| | (thiophene-3-yl)vinyl)benzene-1,3-diol | | |
| 8 | (E)-2-(cyclopent-1-ene-1-yl)-5-(2- | >1 | <50 |
| | (thiophene-3-yl)vinyl)benzene-1,3-diol | | |
| 9 | (E)-2-(cyclopent-1-ene-1-yl)-5-(2- | >1 | <50 |
| | (thiazole-4-yl)vinyl)benzene-1,3-diol | | |
| 10 | (E)-2-(cyclopent-1-ene-1-yl)-5-(2- | >1 | <50 |
| | (thiazole-5-yl)vinyl)benzene-1,3-diol | | |
| 11 | (E)-2-cyclopentyl-5-(2-(thiophene-3- | >1 | <50 |
| | yl)vinyl)benzene-1,3-diol | | |
| 12 | (E)-2-cyclopentyl-5-(2-(thiazole-4- | >1 | <50 |
| | yl)vinyl)benzene-1,3-diol | | |
| 13 | (E)-2-bromo-5-(2-(furan-3- | >1 | <50 |
| | yl)vinyl)benzene-1,3-diol | | |
| 14 | (E)-2-(prop-1-ene-2-yl)-5-(2- | >1 | <50 |
| | (thiophene-3-yl)vinyl)benzene- | | |
| | 1,3-diol | | |
| 15 | (E)-2-bromo-5-(2-(thiophene-3- | >1 | <50 |
| | yl)vinyl)benzene-1,3-diol)-2- | | |
| | (prop-1-ene-2-yl)benzene-1,3-diol | | |
| 16 | (E)-2-(prop-1-ene-2-yl)-5-(2- | >1 | <50 |
| | (thiazole-4-yl)vinyl)benzene-1,3-diol | | |
| 17 | (E)-2-(prop-1-ene-2-yl)-5-(2- | >1 | <50 |
| | (thiazole-5-yl)vinyl)benzene-1,3-diol | | |
| 18 | (E)-2-cyclopropyl-3-methoxy- | >1 | >50 |
| | 5-(2-(thiophene-3-yl)vinyl)phenol | | |
| 19 | (E)-2-bromo-3-methoxy-5-(2- | >1 | >50 |
| | (thiophene-3-yl)vinyl)phenol | | |
| 20 | (E)-5-(2-(furan-3-yl)vinyl)-2- | >1 | >50 |
| | isopropyl-3-methoxyphenol | | |

It can be seen from Table 1 that the novel five-membered heterocycle substituted styrene derivatives synthesized by embodiment 2-20 of the present application have less toxicity to macrophages and higher inhibitory activity against nitric oxide. It is shown that these five-membered heterocycle substituted styrene derivatives with novel chemical structure have low side effects and anti-inflammatory activity, and can be used as new anti-inflammatory drugs to treat various inflammation associated diseases.

As described above, the above embodiments are only used for describing the technical solution of the present application, rather than limiting the same. Although the present application is described in detail by referring to the above embodiments, those ordinary skilled in the art should understand that: the technical solution recorded in each of the above embodiments can be still amended, or some technical features therein can be replaced equivalently. These amendments or replacements do not enable the essence of the corresponding technical solution to depart from the spirit and the scope of the technical solution of each embodiment of the present application.

1. A novel five-membered heterocycle substituted styrene derivative, having a structure shown in formula (I) or a pharmaceutically acceptable salt thereof, a solvent compound of the pharmaceutically acceptable salt, an enantiomer, a diastereoisomer, a tautomer, a racemate or a combination thereof; 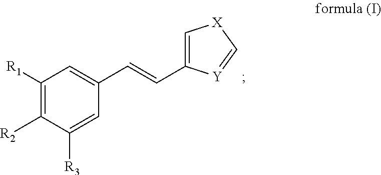
wherein in formula (I), R₁ and R₃ are selected from H, OH, alkoxy or acyl; R₂ is selected from H, alkyl, cycloalkyl, alkenyl, cycloalkenyl, aralkyl or halogen; X is selected from O, S or NR₄, and R₄ is selected from H or alkyl; and Y is selected from C or N.

2. The novel five-membered heterocycle substituted styrene derivative according to claim 1, wherein the pharmaceutically acceptable salt comprises hydrochloride, sulfate, nitrate, phosphate, metaphosphate, mesylate, esilate, citrate, besylate, p-methyl benzenesulfonate, malate, tartrate, succinate, fumarate, acetate, hydroxyacetate, isethionate, maleate, lactate, lactobionate or trifluoroacetate.

3. The novel five-membered heterocycle substituted styrene derivative according to claim 1, wherein the R₁ is selected from OH; the R₃ is selected from OH; the R₂ is selected from propyl; X is selected from O; Y is selected from C; or X is selected from S; and Y is selected from N.

4. The novel five-membered heterocycle substituted styrene derivative according to claim 1, wherein the R₁ is selected from OH; the R₃ is selected from OH; the R₂ is selected from cyclopropyl; X is selected from S; Y is selected from C; X is selected from O; Y is selected from C; or X is selected from S; and Y is selected from N.

5. The novel five-membered heterocycle substituted styrene derivative according to claim 1, wherein the R₁ is selected from OH; the R₃ is selected from OH; the R₂ is selected from cyclopent-1-ene-1-yl; X is selected from S; Y is selected from C; or X is selected from S; and Y is selected from N.

6. The novel five-membered heterocycle substituted styrene derivative according to claim 1, wherein the R₁ is selected from OH; the R₃ is selected from OH; the R₂ is selected from cyclopentyl; X is selected from S; and Y is selected from C.

7. The novel five-membered heterocycle substituted styrene derivative according to claim 1, wherein the R₁ is selected from OH; the R₃ is selected from OH; the R₂ is selected from bromo; X is selected from O; Y is selected from C; X is selected from S; Y is selected from C; or X is selected from S; and Y is selected from C.

8. The novel five-membered heterocycle substituted styrene derivative according to claim 1, wherein the R₁ is selected from OH; the R₃ is selected from OH; the R₂ is selected from prop-1-ene-2-yl; X is selected from S; Y is selected from C; X is selected from O; Y is selected from C; or X is selected from S; and Y is selected from N.

9. A preparation method for the five-membered heterocycle substituted styrene derivative according to claim 1, comprising steps of:
step 1: under the nitrogen protection and the action of catalyst tetrabutylammonium bromide, making a styrene derivative intermediate and triethyl phosphite conduct a first substitution reaction to obtain an ethyl phosphite group substituted styrene derivative;
step 2: under the action of nitrogen protection and alkali reagent sodium hydride, making the styrene derivative with the ethyl phosphite group and a five-membered heterocycle compound conduct a second substitution reaction to obtain a five-membered heterocycle substituted styrene derivative intermediate;
step 3: under the action of aluminum trichloride and N,N-dimethylaniline, removing a benzyl functional group on the five-membered heterocycle substituted styrene derivative intermediate to obtain the five-membered heterocycle substituted styrene derivative;
wherein the styrene derivative intermediate is 3,5-dibenzoxy-4-isopropylbenzyl bromide, 3,5-dimethoxy-4-isopropylbenzyl bromide, 5-(bromomethyl)-2-cyclopropyl-1,3-dimethoxybenzene, 5-bromomethyl-2-(cyclopent-1-ene-1-yl)-1,3-dimethoxybenzene, 5-bromomethyl-2-cyclopentyl-1,3-dimethoxybenzene, 2-bromo-5-(bromomethyl)-1,3-dimethoxybenzene or 5-(bromomethyl)-1,3-dimethoxy-2-(prop-1-ene-2-yl)benzene;
wherein the five-membered heterocycle compound is 4-furfural, thiazole-5-formaldehyde, 3-thenaldehyde, 3-furfural, thiazole-4-formaldehyde, 3,5-dimethoxy-4-(1-methylvinyl)benzaldehyde or 3-methylfuran;
wherein the temperature of the first substitution reaction is 100° C.-150° C., and the time is 4 h-8 h;
wherein the temperature of the second substitution reaction is 20° C.-30° C., and the time is 8 h-16 h.

10. A method for resisting inflammation in a subject in need thereof, comprising administering the five-membered heterocycle substituted styrene derivative according to claim 1 to the subject in need thereof.
